SEQUENCE LISTING (6 sequences)

SEQ ID NO: 1 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - DNA, 1588 bp, Homo sapiens
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcaaaa

SEQ ID NO: 3 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 4 - DNA, 938 bp, Homo sapiens
gcacggaggggcagagaccccggagccccagccccaccatgaccctcggccgccgactcgcgtgtcttttcctcgcctgtgtcctgccggccttgctgctggggggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcgtggcgaatgtaaacgtccgcgcggtgcgggtggtcctgggagcccataacctctcgcggcgggagcccacccggcaggtgttcgccgtgcagcgcatcttcgaaaacggctacgaccccgtaaacttgctcaacgacatcgtgattctccagctcaacgggtcggccaccatcaacgccaacgtgcaggtggcccagctgccggctcagggacgccgcctgggcaacggggtgcagtgcctggccatgggctggggccttctgggcaggaaccgtgggatcgccagcgtcctgcaggagctcaacgtgacggtggtgacgtccctctgccgtcgcagcaacgtctgcactctcgtgaggggccggcaggccggcgtctgtttcggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaataaacattctctgttttgtagaaaaaaaaaaaaaaaa

SEQ ID NO: 5 - protein, 348 aa, Homo sapiens [sequence omitted]

SEQ ID NO: 6 - DNA, 1260 bp, Homo sapiens
  CDS (222)...(1260)
ggggacactggatcacctagtgtttcacaagcaggtaccttctgctgtaggagagagagaactaaagttctgaaagacctgttgcttttcaccaggaagttttactgggcatctcctgagcctaggcaatagctgtagggtgacttctggagccatccccgtttccccgccccccaaaagaagcggagatttaacggggacgtgcggccagagctggggaaatgggcccgcgagccaggccggcgcttctcctcctgatgcttttgcagaccgcggtcctgcaggggcgcttgctgcgttcacactctctgcactacctcttcatgggtgcctcagagcaggaccttggtctttccttgtttgaagctttgggctacgtggatgaccagctgttcgtgttctatgatcatgagagtcgccgtgtggagccccgaactccatgggtttccagtagaatttcaagccagatgtggctgcagctgagtcagagtctgaaagggtgggatcacatgttcactgttgacttctggactattatggaaaatcacaaccacagcaaggagtcccacaccctgcaggtcatcctgggctgtgaaatgcaagaagacaacagtaccgagggctactggaagtacgggtatgatgggcaggaccaccttgaattctgccctgacacactggattggagagcagcagaacccagggcctggcccaccaagctggagtgggaaaggcacaagattcgggccaggcagaacagggcctacctggagagggactgccctgcacagctgcagcagttgctggagctggggagaggtgttttggaccaacaagtgcctcctttggtgaaggtgacacatcatgtgacctcttcagtgaccactctacggtgtcgggccttgaactactacccccagaacatcaccatgaagtggctgaaggataagcagccaatggatgccaaggagttcgaacctaaagacgtattgcccaatggggatgggacctaccagggctggataaccttggctgtaccccctggggaagagcagagatatacgtaccaggtggagcacccaggcctggatcagcccctcattgtgatctgggagccctcaccgtctggcaccctagtcattggagtcatcagtggaattgctgtttttgtcgtcatcttgttcattggaattttgttcataatattaaggaagaggcagggttcaagaggagccatggggcactacgtcttagctgaac

(19) United States
(12) Patent Application Publication
(10) Pub. No.: US 2009/0232910 A1
(43) Pub. Date: Sep. 17, 2009

(54) Biomarkers and therapeutics targets for cognitive decline

(75) Inventors: Donald E. Schmechel, Pittsboro, NC (US); Jeffery N. Browndyke, Pittsboro, NC (US); Kathleen A. Welsh-Bohmer, Durham, NC (US); Kathy L. Sansing-Edwards, Oxford, NC (US)

Correspondence Address: BANNER & WITCOFF, LTD., 1100 13th STREET, N.W., SUITE 1200, WASHINGTON, DC, 20005-4051, US

(73) Assignee: Duke University, Durham, NC (US)

(21) Appl. No.: 11/658,014

(22) PCT Filed: Jul. 22, 2005
(86) PCT No.: PCT/US05/26180

§ 371 Date: Nov. 20, 2008

Related U.S. Application Data
(60) Provisional application No. 60/589,795, filed on Jul. 22, 2004.

(51) Int. Cl. A61K 33/00; C12Q 1/68; G01N 33/00; A61K 38/16; A61K 31/7088; C12Q 1/02; A61K 39/395; C12Q 1/34

(52) U.S. Cl. 424/722; 435/6; 435/7.92; 514/12; 514/44 R; 435/29; 424/130.1; 514/44 A; 435/18

(57) ABSTRACT

Enrichment of S and Z polymorphisms of alpha-1-antitrypsin (AAT) in distinct subsets of patients with cognitive disorder (pre-existing affective disorders and APOE2 allele carriers) suggests that AAT variants are potential endophenotypes for Alzheimer Disease and related disorders of cognition, behavior and affect. Such disorders include ADD/ADHD, learning disabilities, ADEM, and susceptibility to brain injury in toxic/chemical/biological/immunological events. In Alzheimer Disease, S and Z alleles affect age of onset and low AAT levels define faster progression rate. Twenty to thirty percent of all dementia patients display AAT and/or We polymorphisms. Effects of AAT may involve inflammation of liver/lung, macrophage activation and iron and lipid metabolism. AAT, its regulation, and iron metabolism represent therapeutic targets and AAT can serve as a biomarker for vulnerability and disease progression.

This application claims the benefit of U.S. provisional application Ser. No. 60/589,795 filed Jul. 22, 2004, the contents of which are expressly incorporated herein.

This invention was made using funds granted by the United States government from the National Institute of Aging, grant no. 5P50 AG05128-20. The U.S. government retains certain rights in the invention.

### FIELD OF THE INVENTION

The invention relates to the areas of dementias, cognitive disorders and/or affective disorders and/or behavioral dysfunction, including that associated with attention deficit—hyperactivity disorders ‘spectrum’, bipolar disorders, anxiety disorders, and depression, Alzheimer Disease and related dementias. More particularly, it relates to genetic vulnerability, prognostic methods, treatment methods, and drug screening methods.

### BACKGROUND OF THE INVENTION

The gene for the acute phase protein alpha-1-antitrypsin (AAT; also known as serpinA1 and Pi) lies in the serpin gene cluster on chromosome 14q32.1. S and Z polymorphic alleles of the AAT gene are commonly encountered in persons of Western European origin, usually combined with common M subtypes (M, M1, M2) (1-3). MS and MZ are clinically silent, but MZ is associated with lower AAT levels. Other more rare ‘deficiency’ polymorphisms have been described, and similar alleles have been described or remain to be discovered in non-European populations. Deficiency polymorphisms may impact on susceptibility to liver or pulmonary infections, and outcome of substance abuse, or environmental exposure affecting liver or lung. A possible relation to Alzheimer Disease (AD) has been noted for serpin enzyme complex receptor (4), presence of AAT in astrocytes and plaques (5), elevated serum levels (6), and inverse relation of cerebrospinal fluid AAT and oxidized LDL (7). In primates, liver abnormalities including hepatosteatosis and mitochondrial dysfunction associated with iron overload and copper deficiency correlate with eventual AD-like brain pathology, as well as abnormalities in beta-islet cells of pancreas, kidney proximal epithelial cells, cardiac myocytes, and choroid epithelia. (8).

Other hepatic-expressed genes include apolipoprotein E (APOE) alleles—polymorphisms 2, 3 and 4 coded on chromosome 19 (9). Polymorphisms or mutations of hemochromatosis gene (Hfe) C282Y, H63D and S65C are coded for on chromosome 6 (10). We have found that excessive iron stores with copper deficiency states in a primate model of aging result in mitochondrial damage, macrophage activation, accelerated aging of heart, pancreas, kidney and brain (multiple systems) and AD pathology (8, 11).

Accelerated autophagy and mitochondrial injury is described with AAT deficiency polymorphisms (12). Lower AAT levels have been associated with accelerated rates of coronary artery disease (13). AAT and one of its substrates, neutrophil elastase, have been connected to regulation of intra- and extracellular iron metabolism, and to regulation of cholesterol metabolism as part of the acute phase reactant system and innate immunity (14).

There is a need in the art for additional tools for prognosticating and treating dementias, including Alzheimer Disease and various disorders of cognitive, affective and/or behavioral dysfunction. There is a need in the art for means of identifying new therapeutic agents for treating dementias, cognitive dysfunction, and the above-mentioned, related disorders.

### BRIEF SUMMARY OF THE INVENTION

A method is provided for predicting rate of progression of cognitive and/or behavioral decline in a subject with attention deficit disorder/attention deficit hyperactivity disorder, an affective disorder, mild cognitive impairment, dementia, or Alzheimer Disease. Types of alleles of alpha-1-antitrypsin (AAT) or AAT level in the subject with attention deficit disorder/attention deficit hyperactivity disorder, an affective disorder, mild cognitive impairment, dementia, or Alzheimer Disease is determined. The determined types of alleles and/or AAT level are used as a factor to predict rate of progression of cognitive and/or behavioral decline in the subject.

A method is also provided for predicting vulnerability to or age of onset of attention deficit disorder/attention deficit hyperactivity disorder, an affective disorder, mild cognitive impairment, dementia, or Alzheimer Disease. Types of alleles of alpha-1-antitrypsin (AAT) or AAT level in a subject are determined. The determined types of alleles and/or AAT level are used as a factor to predict vulnerability to or age of onset of Alzheimer Disease.

An additional method is provided for predicting persons at risk of long-term nervous system injury. Types of alleles of alpha-1-antitrypsin (AAT) or AAT level in a subject who has been exposed to a neurotoxic or neuroinflammatory agent is determined. The determined types of alleles or AAT level are used as a factor to predict persons at risk of long-term nervous system injury.

Also provided by the present invention is a method for predicting vulnerability to nervous system injury or cognitive or affective disorder of persons with pulmonary disease, liver disease, or coronary artery disease. Types of alleles of alpha-1-antitrypsin (AAT) or AAT level in a subject with pulmonary disease, liver disease, or coronary artery disease are determined. The determined types of alleles or AAT level are used as a factor to predict vulnerability of the subject to nervous system injury or cognitive or affective disorder.

A method is provided of delaying age of onset or progression rate of cognitive dysfunction in a subject at risk of developing cognitive dysfunction or with cognitive dysfunction. AAT protein or a nucleic acid encoding AAT protein or C282Y hemochromatosis protein or a nucleic acid encoding C282Y hemochromatosis protein is administered to the central nervous system of the subject. Level or activity of AAT protein or C282Y protein in the central nervous system of the subject is thereby increased.

Another method is provided of delaying age of onset or progression rate of cognitive dysfunction in a subject. AAT protein or a nucleic acid encoding AAT protein or C282Y hemochromatosis protein or a nucleic acid encoding C282Y hemochromatosis protein is administered to the blood stream or liver of a subject at risk of developing Alzheimer Disease. Level or activity of AAT protein or C282Y protein in the blood stream or liver of the subject is thereby increased.

According to another aspect of the invention a method is provided of delaying age of onset or diminishing progression rate of cognitive dysfunction in a subject who has an AAT deficiency phenotype. Thiamine supplements or a low carbohydrate diet is administered to the subject. Age of onset of cognitive dysfunction is thereby delayed or progression rate of cognitive dysfunction is diminished.

A method is also provided of diminishing progression rate of cognitive dysfunction in a subject who has attention deficit disorder, an affective disorder, or Alzheimer Disease. Thiamine supplements or a low carbohydrate diet is administered to the subject. Age of onset of cognitive dysfunction is thereby delayed or progression rate of cognitive dysfunction is diminished.

Also provided is a method of screening for candidate drugs for treatment of cognitive dysfunction. A test substance is contacted with an AAT protein encoded by an S, Z, I, P, F, V, G, or null allele. Activity of the AAT protein is determined. The test substance is identified as a candidate drug for treatment of cognitive dysfunction if it increases activity of the AAT protein.

Another aspect of the invention is a method of screening for candidate drugs for treatment of cognitive dysfunction. A test substance is contacted with a cell which has an AAT-deficient phenotype. Activity of AAT protein in the cell is determined. The test substance is identified as a candidate drug for treatment of cognitive dysfunction if it increases total amount of activity of AAT in the cell.

Another aspect of the invention is a method of screening for candidate drugs for treatment of cognitive dysfunction. A test substance is contacted with a cell which encodes an AAT protein. Amount of AAT protein expressed in the cell is determined. The test substance is identified as a candidate drug for treatment of cognitive dysfunction if it increases expression or activity of AAT in the cell.

Another aspect of the invention is method of screening for candidate drugs for treatment of cognitive dysfunction. A test substance is contacted with an AAT protein encoded by an S, Z, I, P, F, V, G, or a null allele. The AAT is contacted with cells selected from the group consisting of monocytes, macrophages, astroglia, microglia, oligodendroglia, choroid plexus cells, cerebral endothelial cells, and progenitors thereof. A parameter selected from the group consisting of ferritin concentration, transferrin (Tf) receptor concentration, endocytosis of Tf receptor and ligand, free iron concentration, IRE/IRP activity, and IRE/IRP modulated targets is determined in the cells. The test substance is identified as a candidate drug for treatment of cognitive dysfunction if it increases the parameter.

Another aspect of the invention is a method of screening for candidate drugs for treatment of cognitive dysfunction. A test substance is contacted with a first cell which has an AAT-deficient phenotype. The AAT from the cell is contacted with second cells selected from the group consisting of monocytes, macrophages, astroglia, microglia, oligodendroglia, choroid plexus cells, cerebral endothelial cells, and progenitors thereof. A parameter selected from the group consisting of ferritin concentration, transferrin (Tf) receptor concentration, endocytosis of Tf receptor and ligand, free iron concentration, IRE/IRP activity, and IRE/IRP modulated targets is determined in the second cells. The test substance is identified as a candidate drug for treatment of cognitive dysfunction if it increases the parameter.

Another aspect of the invention is a method of screening for candidate drugs for treatment of cognitive dysfunction. A test substance is contacted with a first cell which encodes an AAT protein. The AAT from the first cell is contacted with a second cell selected from the group consisting of monocytes, macrophages, astroglia, microglia, oligodendroglia, choroid plexus cells, cerebral endothelial cells, and progenitors thereof. A parameter selected from the group consisting of ferritin concentration, transferrin (Tf) receptor concentration, endocytosis of Tf receptor and ligand, free iron concentration, IRE/IRP activity, and IRE/IRP modulated targets is determined in the second cell. The test substance is identified as a candidate drug for treatment of cognitive dysfunction if it increases the parameter.

Another aspect of the invention is method of screening for candidate drugs for treatment of cognitive dysfunction. A test substance is contacted with an AAT protein encoded by an S, Z, or other deficiency allele or a null allele. Carbonyl or oxidized or nitrosylated groups on the AAT protein or activity of the AAT protein is determined. The test substance is identified as a candidate drug for treatment of cognitive dysfunction if it modulates amount of carbonyl or oxidized or nitrosylated groups on the AAT protein and/or activity of AAT protein.

Another aspect of the invention is method for screening for candidate drugs for treatment of cognitive dysfunction. A test substance is contacted with a cell which has an AAT-deficient phenotype. Amount of carbonyl or oxidized or nitrosylated groups on the AAT protein in the cell or the activity of the AAT protein is determined. The test substance is identified as a candidate drug for treatment of cognitive dysfunction if it modulates the amount of carbonyl or oxidized or nitrosylated groups on the AAT protein and/or activity of AAT protein.

Another aspect of the invention is a method for screening for candidate drugs for treatment of cognitive dysfunction. A test substance is contacted with a cell which encodes an AAT protein. Amount of carbonyl or oxidized or nitrosylated groups on the AAT protein expressed in the cell or activity of the AAT protein is determined. The test substance is identified as a candidate drug for treatment of cognitive dysfunction if it modulates amount of carbonyl or oxidized or nitrosylated groups of AAT and/or activity of AAT.

Yet another aspect of the invention provides a method of screening for candidate drugs for treatment of cognitive dysfunction. A test substance is contacted with an AAT protein encoded by an S, Z, or other deficiency allele or a null allele. Monocyte and/or macrophage activating activity of the AAT protein are determined. The test substance is identified as a candidate drug for treatment of cognitive dysfunction if it increases monocyte and/or macrophage activating activity of the AAT protein.

Another aspect of the invention is a method of screening for candidate drugs for treatment of cognitive dysfunction. A test substance is contacted with a cell which has an AAT-deficient phenotype. Monocyte and/or macrophage activating activity of the AAT protein are determined. The test substance is identified as a candidate drug for treatment of cognitive dysfunction if it increases monocyte and/or macrophage activating activity of the AAT protein.

Another aspect of the invention is a method for screening for candidate drugs for treatment of cognitive dysfunction. A test substance is contacted with a cell which encodes an AAT protein. Monocyte and/or macrophage activating activity of the AAT protein are determined. The test substance is identified as a candidate drug for treatment of cognitive dysfunction if it increases monocyte and/or macrophage activating activity of the AAT protein.

Another aspect of the invention is a method of screening for candidate drugs for treatment of cognitive dysfunction. A test substance is contacted with an AAT protein encoded by an S, Z, or other deficiency allele or a null allele. Amount of carboxyl terminal fragment of AAT (C-36) is determined. The test substance is identified as a candidate drug for treatment of cognitive dysfunction if it modulates amount of the C-36.

Another aspect of the invention is a method of screening for candidate drugs for treatment of cognitive dysfunction. A test substance is contacted with a cell which has an AAT-deficient phenotype. Amount of carboxyl terminal fragment of AAT (C-36) is determined. The test substance is identified as a candidate drug for treatment of cognitive dysfunction if it modulates amount of the C-36.

Another aspect of the invention is a method for screening for candidate drugs for treatment of cognitive dysfunction. A test substance is contacted with a cell which encodes an AAT protein. Amount of carboxyl terminal fragment of AAT (C-36) is determined. The test substance is identified as a candidate drug for treatment of cognitive dysfunction if it modulates amount of the C-36.

Another aspect of the invention is a method of delaying age of onset or progression rate of cognitive dysfunction in a subject having or at risk of developing attention deficit disorder/attention deficit hyperactivity disorder, an affective disorder, mild cognitive impairment, dementia, or Alzheimer Disease. An agent for inhibiting neutrophil elastase activity or expression is administered to the central nervous system of the subject. The agent is selected from the group consisting of: an antibody which specifically binds to neutrophil elastase, an antisense molecule comprising at least 18 contiguous nucleotides which are complementary to mRNA encoding neutrophil elastase, FR901277, SC-37698, SC-39026, SKALP/elafin, pre-elafin (68), SLPI (69), sivelestat (ONO-5046; Elaspol; C₂₀H₂₁N₂O₇S.4H2O.Na.), ONO-6818 (C₂₃H₂₈N₆O₄, molecular weight: 452.51)), FR901277 (C₄₇H₆₃N₉O₃, molecular weight: 961), SC-37698 (Searle, Skokie, Ill.), SC-39026 (Searle, Skokie, Ill.), and SSR69071 (2-(9-(2-Piperidinoethoxy)-4-oxo-4H-pyrido[1,2-a]pyrimidin-2-yloxy-methyl)-4-(1-methylethyl)-6-methoxy-1,2-benzisothiazol-3(2H)-one-1,1-dioxide). Age of onset or progression rate of cognitive dysfunction in the subject is delayed or diminished.

Another aspect of the invention is a method of screening for candidate drugs for treatment of cognitive dysfunction. A test substance is contacted with a neutrophil elastase protein. Activity of the neutrophil elastase protein is determined. The test substance is identified as a candidate drug for treatment of cognitive dysfunction if it decreases activity of the neutrophil elastase protein.

Another aspect of the invention is a method of screening for candidate drugs for treatment of cognitive dysfunction. A test substance is contacted with a cell which expresses a human neutrophil elastase. Activity of human neutrophil elastase protein in the cells determined. The test substance is identified as a candidate drug for treatment of cognitive dysfunction if it decreases total amount of activity of the neutrophil elastase in the cell.

Another aspect of the invention is a method of delaying age of onset or diminishing progression rate of cognitive dysfunction in a subject who has an AAT deficiency phenotype. Lithium is administered to the subject. Age of onset of cognitive dysfunction is thereby delayed or progression rate of cognitive dysfunction is thereby diminished.

Another aspect of the invention is a method of diminishing progression rate of cognitive dysfunction in a subject who has attention deficit disorder, an affective disorder, or Alzheimer Disease. Lithium is administered to the subject. Progression rate of cognitive dysfunction is thereby diminished.

### BRIEF DESCRIPTION OF THE DRAWINGS

FIG. 1 presents a Kaplan-Meier curve of the effect of AAT polymorphisms on age of onset for persons presenting with cognitive, affective or behavioral disorders. Age of onset for cognitive symptoms according to AAT polymorphisms with M signifying various M, M1, M2 combinations, S signifying MS heterozygote and Z signifying MZ heterozygote. Difference is significantly earlier age of onset, p=0.02 (Log rank), for MZ versus other phenotypes.

FIG. 2 presents Kaplan-Meier curve for effect on age of onset of low vs. normal expression levels of AAT in serum (regardless of AAT polymorphism). These data indicate that other polymorphisms which cause low expression will have the same effect. Age-of-onset curves for set of persons with AAT polymorphisms associated with deficiency (MS, MZ) regardless of AAT level combined with normal phenotypes or alleles (M subclasses) with low level (<110 mg/dL). Earlier onset is significant (p=0.0004, Log rank).

FIG. 3 shows change in mini-mental status exam (MMSE) scores for persons with early Alzheimer Disease (AD) compared to level of AAT at time of presentation (p=0.28, p=0.04, uncorrected for C282Y status, APOE genotype, gender, age of onset). Higher AAT levels are associated with slower rate of change of MMSE scores (more stable course).

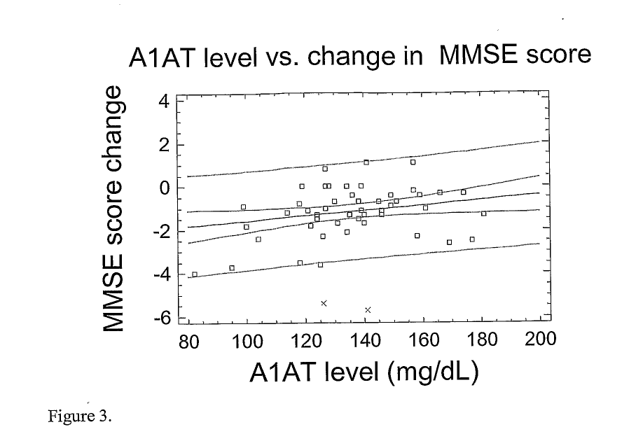

FIG. 4 shows the association of AAT levels and affective disorder.

FIG. 5 shows AAT levels of untreated versus treated patients with bipolar disorder.

FIG. 6 shows free copper versus duration in AD group.

FIG. 7 shows age of onset versus transferrin quartiles (AD/MCI).

FIG. 8 shows A1AT level versus change in MMSE.

FIG. 9 shows MMSE change (points per year).

FIG. 10 shows gender versus change in MMSE.

FIG. 11 shows C282Y vs. change in MMSE.

FIG. 12 shows APOE43 alleles versus change in MMSE.

### DETAILED DESCRIPTION OF THE INVENTION

It is a discovery of the present inventors that certain biochemical and genetic markers can be used as predictors of progress, onset and vulnerability to dementias, cognitive dysfunction in affective disorders such as bipolar disorder spectrum, anxiety disorders, attention deficit disorders such as ADD and ADHD, and to cerebral injury particularly to white matter such as post-immunization reactions or after toxic infectious, chemical or biological exposures. Certain alleles or polymorphisms and/or expression levels of the alpha-1-antitrypsin (AAT) gene are associated with these effects on age of onset, progression rate and vulnerability. Certain alleles of the apolipoprotein E (APOE) gene and of the hemochromatosis gene (Hfe) interact with the AAT gene to influence age of onset, progression rate and vulnerability. Other relevant factors include age of onset, gender, bound and free iron, serum transferrin and transferrin receptor levels, c-reactive protein, serum fibrinogen, bound and free copper, and ceruloplasmin (ferroxidase) activity, levels and expression. High AAT levels are associated with later age of onset, slower progression rate, and diminished vulnerability.

Main ancillary genetic factors include number of APOE4 alleles; later age of onset, slower progression rate, and reduced vulnerability are associated with fewer APOE4 alleles. Our data support an effect of C282Y hemochromatosis polymorphism with later age of onset, slower progression rate, and reduced vulnerability. Other important ancillary and associated biochemical factors include C-reactive protein levels (lower level is stabilizing of patient condition), fibrinogen (higher level is stabilizing), and serum transferrin (lower level is stabilizing). Elevated levels of transferrin and/or C-reactive protein are additional negative prognostic indicators. Levels of such proteins or metabolites can be determined by any method known in the art.

AAT alleles S, Z, I, P, F, G, V or null (or similar ‘deficiency’ alleles in non-Caucasian populations) are poor prognostic factors (i.e., they indicate a poor prognosis) as is a lowered anti-proteolytic activity or level or release of AAT. A subject's genotype or phenotype can be determined by any method known in the art. These include determining the sequence of the AAT alleles in the person, performing a sequence based test, such as a test employing an allele-specific oligonucleotide hybridization or allele-specific amplification, electrofocusing of proteins, restriction fragment length polymorphism, dual-color detection by ligase-mediated analysis, temperature or denaturing gradient gel electrophoresis, anti-proteolytic activity determinations, specific ELISA methods, or PCR-mediated site-directed mutagenesis. Any technique known in the art for determining an AAT genotype or phenotype can be used. See for example Lucotte G, et al., “Polymerase chain reaction detection of S and Z alpha-1-antitrypsin variants by duplex PCR assay.” Mol Cell Probes. 1999; 13:389-91. See also Brantly M, et al., “Molecular basis of alpha-1-antitrypsin deficiency.” Am J Med 1988; 84:13-31; Nukiwa et al. Characterization of the M1(ala²¹³) type of α1-antitrypsin haplotype. Biochemistry 1987; 26:5259-5267; Hejtmancik et al. “Prenatal diagnosis of alpha 1-antitrypsin deficiency by restriction fragment length polymorphisms, and comparison with oligonucleotide probe analysis.” Lancet 1986; 2:767-770; Klasen et al. “Detection of alpha-1-antitrypsin deficiency variants by synthetic oligonucleotide hybridization.” Clin Chim Acta 1987; 170:201-207; Newton et al. “Analysis of any point mutation in DNA. The amplification refractory mutation system (ARMS).” Nucleic Acids Res 1989; 17:2503-2516; Samiotaki et al. “Dual-color detection of DNA sequence variants by ligase-mediated analysis.” Genomics 1994; 20:238-242; Johnson et al. “Detection of the common alpha-1-antitrypsin variants by denaturing gradient gel electrophoresis.” Ann Hum Genet 1991; 55:183-198; Lam et al. “Rapid screening for α₁-antitrypsin Z and S mutations.” Clin Chem 1997; 403-404; Braun et al. “Rapid and simple diagnosis of the two common α₁-proteinase inhibitor deficiency alleles Pi*Z and Pi*S by DNA Analysis.” Eur J Clin Chem Clin Biochem 1996; 34:761-764; Hammerberg et al. “Polymerase chain reaction-mediated site-directed mutagenesis detection of Z and S alpha-1-antitrypsin alleles in family members.” J Clin Lab Anal 1996; 10:384-388. Other genetic polymorphisms in apolipoprotein E and the hemochromatosis gene are identified as described below and according to a variety of well-established methods for detecting single-site polymorphisms.

Rate of future cognitive decline in persons with early stages (mini-mental status exam or MMSE score >23) of AD and related dementias can be calculated using some or all of the following factors: age of onset, sex, clinical diagnosis, apolipoprotein E allele type, and hemochromatosis allele type. Presence of 1-2 APOE4 alleles weakly accounts for more rapid decline. Presence of 1-2 hemochromatosis alleles C282Y strongly accounts for less rapid decline (C282Y acts as a stabilizing gene). Major factors (since APOE4 is over-represented and commonly present and C282Y is present in only 10-15% of cases) are A1AT alleles and particularly expression level in serum), C-reactive protein levels (high sensitivity assay), and/or copper status (determined either by free copper, ceruloplasmin or copper/zinc ratio). A logistic regression analysis employing these variables can provide coefficients and predict categories of stability or decline or actual MMSE change with high sensitivity and specificity (>80%) with cut-points determined by discriminant analysis. Increased inflammatory ‘state’ and levels of A1AT compared to background assessment of inflammatory drive (lower levels of C-reactive protein) is also associated with slower progression and stability. This analysis can be used to stratify patients to improve efficiency of drug studies, for clinical classifications, for prognosis of AD and related disorders. Rates of cognitive decline in normal individuals may be accounted for by same variables and approach.

Patients who can be tested and/or treated according to any of the methods of the present invention include those who present with cognitive dysfunction with a history of treated depression, cognitive dysfunction with a history of depression, cognitive dysfunction with bipolar disease or schizoaffective disorders, cognitive dysfunction with generalized anxiety disorder, cognitive dysfunction with attention deficit, ADHD disorder or both attention deficit and ADHD disorder, dyslexia, developmental delay, school adjustment reaction, Alzheimer Disease, amnesic mild cognitive impairment, non-amnesic mild cognitive impairment, cognitive impairment with white matter disease on neuroimaging or by clinical examination, frontotemporal dementia, cognitive disorders in those under 65 years of age, those with serum homocysteine levels of less than 10 nmol/l, and those with high serum transferrin levels (uppermost population quartile).

Patients who can be tested and/or treated also include those who present with previous or present physical illnesses associated with cardiac, pulmonary, infectious or hepatic events or dysfunction whether congenital, acquired, infectious, or toxic chemical or biological, with physical illnesses or history of post-immunization reactions, with mixed medical-psychiatric illnesses such as chronic fatigue syndrome, fibromyalgia, post-traumatic stress disorder, substance abuse (alcoholism, etc) who may present with or may have cognitive, affective or emotional components to their illness. These patients will benefit from testing and analysis with regard to AAT status and associated markers.

Patients who are at risk of developing neurological damage and/or Alzheimer Disease and/or affective disorder include those with family histories, those with AAT deficiency alleles, those with low serum levels of AAT, and those who have been exposed to neurotoxic or neuroinflammatory agents. Such agents include multiple immunizations, chemical or biological weapons, and infectious agents.

Specific alleles of other hepatic-associated genes (apolipoprotein E or APOE and hemochromatosis or Hfe) are also negative prognostic indicators. For example, the APOE4 allele in combination with certain AAT alleles and the wild-type allele of the Hemochromatosis gene are negative prognostic indicators. C282Y polymorphism (mutation) of the hemochromatosis gene is a positive prognostic and protective factor.

AAT protein or a nucleic acid encoding AAT protein or C282Y hemochromatosis protein or a nucleic acid encoding C282Y hemochromatosis protein or substances or therapies aimed at promoting release or effective activity of AAT can be administered to subjects described above to avoid or delay the onset or reduce the progression rate of cognitive and/or affective and/or behavioral dysfunction. These can be delivered intrathecally directly into the brain, intracerebroventricularly, intravenously, transdermally, via pump/bypass technology, by cell implants, or directly to the liver or other relevant organs. Methods of delivering proteins and nucleic acids are well known in the art, and any such methods can be used. These include formulations in liposomes, in microparticles, complexed with carrier proteins or polymers, in viral vectors, in plasmid vectors, or fixed to solid or soluble substrates. Exemplary sequences for human ATT are shown in SEQ ID NOs: 1 and 2. Any allelic versions can be used. Typically, these are at least 95%, 96%, 97%, 98%, and 99% identical to the sequences shown. Exemplary sequences for human C282Y are shown in SEQ ID NOs: 5 and 6. There are many isoforms known; isoforms with the C282 polymorphism/mutation can be used as well.

Thiamine can also be administered to subjects with or without an AAT deficiency phenotype or with or without AAT deficiency alleles. Such administrations will delay the onset or reduce the progression rate of cognitive dysfunction. Thiamine can be administered by any means known in the art, including intramuscular, intravenous, and oral routes. Any method for treating insulin resistance, reactive hypoglycemia, or hepatosteatosis/liver dysfunction by exercise, diet and/or pharmacological means is also beneficial in those persons identified as being at risk or affected.

Candidate drugs for treating cognitive and/or affective and/or behavioral dysfunction can be identified using simple biochemical or cell-based tests. In one test, an AAT protein encoded by a deficiency allele, such as an S or Z allele, is contacted with a test substance. Activity of the contacted protein is determined. If a test substance increases the activity of the protein, it is identified as a candidate drug. Any format known in the art can be used for testing the AAT protein's antiproteolytic activity. Other tests can be performed to confirm the usefulness of the test substance for treatment of humans. If a test substance increases ability of AAT to activate monocytes/macrophages/microglia, it is identified as a candidate drug. Candidate drugs identified for treatment of AAT deficiency-associated illnesses, such as chronic obstructive pulmonary disease and/or liver dysfunction and/or pancreatic dysfunction, are candidates for use in the treatment/prevention of the disorders mentioned above. Such candidate drugs can be further tested using other methods described herein. Some assays according to the invention may detect both increases and decreases in amount or activity of AAT and its modified products. Candidates which decrease AAT may also be useful to obtain homeostasis in patients. Modulation of amount or activity encompasses both increases and decreases.

Neutrophil elastase (HNE) is a target for AAT proteolytic action. Neutrophil elastase sequences are shown in SEQ ID NOs: 3 and 4. The signal peptide consists of amino acid residues 1-29. The proprotein consists of amino acid residues 30-267. The mature protein consists of amino acid residues 30-247. Allelic forms of neutrophil elastase can be used which are at least 95%, 96%, 957%, 98%, 99% identical to the sequences shown. Antibodies can be generated which specifically bind to HNE according to methods which are well known in the art. The antibodies can be polyclonal or monoclonal. Preferably they will not bind appreciably to other human proteins. Preferably their affinity for HNE will be at least 10, at least 100, or at least 1000 fold stronger for HNE than for other human proteins. Antisense molecules can also be used to inhibit HNE expression. Such molecules typically will comprise at least 18, 20, 22, 24, or 26 contiguous nucleotides which are complementary to mRNA encoding HNE. Typically antisense molecules will be designed to hybridize to the 5′ half, quarter, or third of the mRNA. Small molecules may also be used to inhibit HNE. Various inhibitors are known in the art and any of these can be used. Inhibitory RNA (RNAi) can also be used. Such RNA is typically double stranded at ranges from about 20 bp to 35 bp. Often a two-base, 3′ overhang is used. Design guidelines for RNAi are known in the art. See, e.g., Integrated DNA Tecnologies, Inc. “Dicer Substrate RNAi Design” at the idtdna.com site. Other inhibitors known in the art which may be used include FR901277, SC-37698, SC-39026, SKALP/elafin (68), pre-elafin, SLPI (69), sivelestat (ONO-5046; Elaspol; C₂₀H₂₁N₂O₇S.4H2O.Na.), ONO-6818 (C₂₃H₂₈N₆O₄, molecular weight: 452.51)), FR901277 (C₄₇H₆₃N₉O₁₃, molecular weight: 961), SC-37698 (Searle, Skokie, Ill.), SC-39026 (Searle, Skokie, Ill.), and SSR69071 (2-(9-(2-Piperidinoethoxy)-4-oxo-4H-pyrido[1,2-a]pyrimidin-2-yloxy-methyl)-4-(1-methylethyl)-6-methoxy-1,2-benzisothiazol-3(2H)-one-1,1-dioxide).

HNE can be used as a target for drug development, i.e., by screening for substances which decrease its activity or expression. Such drugs can be used to treat cognitive dysfunction. Cell based or in vitro assays can be used to screen for inhibitors of HNE.

Lithium, primarily lithium carbonate, is currently used to treat affective disorders such as bipolar disease. Lithium can also be used to delay the onset of cognitive dysfunction or to diminish the progression rate of cognitive dysfunction. Patients to be so treated include those with an AAT deficiency phenotype or genotype, as well as those who are at risk of developing ADD/ADHD, an affective disorder, or Alzheimer's disease. Dosage, formulations, and administration routes can be used within known guidelines for other indications, using AAT levels as an additional therapeutic marker.

Candidate drugs can also be tested in cell-based tests. In one such test a cell with one or more AAT deficiency alleles is contacted with a test substance. If the test substance increases the anti-proteolytic activity or modifies post-translational structure or properties of AAT protein in the cell so that its ability to activate monocytes and macrophages is increased, the test substance is identified as a possible drug for preventing or retarding cognitive and/or affective and/or behavioral dysfunction.

Another cell based test measures the amount of AAT expressed by a cell after being contacted with a test substance. If the test substance increases the expression of AAT or modifies post-translational structure or properties of AAT protein so that its ability to activate monocytes and macrophages is increased, then it is identified as a candidate for treatment, delay, or reduction of cognitive and/or affective and/or behavioral dysfunction. Modified AAT protein structures which can also be measured include oxidized/nitrosylated forms and cleaved forms, in particular, a fragment of AAT that consists of the carboxyl terminal residues 259-394. Increased amounts, activity, or release of any of these forms of AAT can be determined.

Oxidation/nitrosylation of AAT hinders its ability to interact with most serine proteases, including pancreatic elastase. Oxidized AAT increases the production of superoxide by monocytes and/or macrophages. Superoxide generation can be assayed by monitoring ferricytochrome C reduction. See Moraga et al., J. Biol. Chem. 275, 7695-7700, 2000. Another method is to measure superoxide adducts.

The C-terminal fragment of AAT (proteolytically cleaved C-36) mediates pro-inflammatory activation of monocytes and macrophages. See Moraga et al, supra, Janciauskiene et al., Scand. J. Clin. Lab Invest. 57, 325, 336, 1997; Janciauskiene et al., Eur. J. Biochem. 254, 460-467, 1998; Janciauskiene et al., Hepatology 29, 434-442, 1999; Janciauskiene et al., Atherosclerosis 147, 263-275, 1999. The generation of pro-inflammatory cytokines and chemokine (MCP-1) can be assayed as an indicator of monocyte and macrophage activation. Any means of assaying for pro-inflammatory cytokines and chemokines can be used. For example, a sandwich enzyme immunoassay can be used to quantitate cytokines such as IL-6 and TNFα, and chemokines such as MCP-1. See Janciauskiene et al., Atherosclerosis, 158, 41-51, 2001. Production of human gelatinase B and MMP-9, increased uptake of LDL, as well as oxygen consumption can be measured and used as indicators of activation of monocyte and macrophages. Any means known in the art for measuring and determining monocyte and macrophage activation can be used in the invention.

AAT significantly increases ferritin concentration in monocytes and macrophages. See Graziadei, et al., Exp. Hematol. 26, 1053-1060, 1998. Ferritin can be measured conveniently by means of an enzyme-linked immunoadsorbent assay (ELISA). Transferrin receptor concentration, endocytosis rates, extracellular and intracellular ferritin concentration, free iron, iron related proteins (IRP1 and IRP2), and iron related elements (IRE) can be measured by any means known in the art for protein, mRNA, etc. (see references).

AAT alleles and/or expression level can be determined in persons newly presenting with or at risk of the cognitive disorders listed above. Deficiency alleles and/or low expression levels indicate increased vulnerability to anxiety or mood disorder, hepatic/immunological vulnerability, dyslexia, developmental disorders, and cerebral white matter damage such as vascular injury, ischemia, and dysmyelination/demyelination. Subtyping of pre-existing ADD/ADHD, anxiety or bipolar disorders can also be performed using these determinations, for example, for purposes of clinical testing, research, and choosing treatment options. According to the present invention, low levels of AAT are those <110 mg/dL with normal c-reactive protein <0.20 mg/dL or <120 mg/dL with c-reactive protein >=0.20 mg/dL.

These determinations can be used to predict vulnerability to hepatotoxicity or immunotoxicity of therapeutic approaches, predicting present and future vulnerability to white matter disease (including post-immunization reactions such as in multiple immunizations, for example, of prenatal or postnatal children during CNS development, or for deployed personnel, for ‘vascular’ white matter or neuronal injury associated with fever, encephalomyelitis, hypertension, diabetes, dyslipidemias), predicting present and future vulnerability to infectious, toxic chemical or biological exposures affecting the central or peripheral nervous system, for clinical detection and management of possible associated reactive hypoglycemia, hepatosteatosis, metabolic syndrome X and thiamine or other nutritional deficiencies, and for estimating rate of decline in AD or related disorders. For example, vulnerability to immunological based therapies with potential for vasculitic or encephalitic complications (e.g., passive and active immunization for beta-amyloid therapies) can be predicted and dosage or regimen choice selected according to AAT phenotype and levels.

Likewise, AAT can be used as a biomarker for vulnerability to anxiety, mood disorder or cognitive impairment in persons presenting with pre-existing conditions associated with AAT deficiency such as reactive airway disease, chronic obstructive pulmonary disease, coronary artery disease and liver disease.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

### EXAMPLES

### Example 1

### AAT Levels

Average level of serum AAT was 136±24 mg/dL in 918 persons (normal 100-190 mg/dL, SI conversion 0.184 micromol/L). Higher levels correlate with age (p<0.01), with female gender (p<0.0001; 140±24 vs. 132±25), and AAT phenotype (p<0.0001; with M>S>>Z levels: 140±24, 122±22, 88±18). Levels for AD vs. non-AD categories did not vary (p=0.55) and were significantly higher than normal/CIND (cognitive impairment, no dementia) group (p=0.06) [data not shown]. AAT level was low in 100 of 918 persons (<110 mg/dL) and in 15 persons >200 mg/dL. Distribution of low levels was 6% of persons with MM phenotype, 31% of MS, and 84% of MZ phenotypes. When considered as a whole, AAT levels correlated weakly with acute phase reactants/indices such as serum ceruloplasmin, copper, and copper/zinc ratio (r=0.35-0.42, r2=0.14, p<0.0001) and iron (r=−0.18, p<0.001) and not at all with serum ferritin, transferrin, c-reactive protein or plasma fibrinogen. In 245 cases, two AAT levels were available (6 months to two years apart) and showed stability (r=0.69, r2=0.48, p<0.00001) similar to ceruloplasmin (n=286 patients, r=0.70, r2=0.49, p<0.00001, average interval=1.7 years).

### Example 2

### AAT Phenotypes and Subsets

In all persons presenting at risk or with cognitive complaints/disorders (table below), the proportion of non-MM phenotypes was slightly increased (ca. 14%) compared to expected 9% (reference 3, table 1). This difference may be due to the wide variation across different regions of the United States.

Frequency Table for AAT polymorphisms and diagnostic family

| AAT | | | | |
|---|---|---|---|---|
| M . . . | Normal/CIND | AD Related | Non-AD | Totals |
| MM | 75 | 510 | 220 | 805 |
| | 84.3% | 86.0% | 87.0% | 86.1% |
| MS | 7 | 59 | 20 | 86 |
| | 7.9% | 10.0% | 7.9% | 9.2% |
| MZ | 5 | 15 | 11 | 31 |
| | 5.6% | 2.5% | 4.4% | 3.3% |
| Other | 2 | 9 | 2 | 13 |
| Alleles | 0.2% | 1.5% | 0.7% | 1.4% |
| Column | 89 | 593 | 253 | 935 |
| Totals | | | | |

In all persons presenting at risk or with cognitive complaints/disorders, the proportion of persons with pre-existing affective disorders is shown in table below and represents from 25-50% of persons in that diagnostic category.

Frequency Table for presence of affective
disorders and diagnostic family

| Affective | | | | |
|---|---|---|---|---|
| Disorder | Normal/CIND | AD Related | Non-AD | Totals |
| None | 49 | 450 | 184 | 683 |
| | 55.1% | 75.8% | 73.3% | |
| Depression | 16 | 58 | 28 | 102 |
| | 18.0% | 9.8% | 11.2% | |
| Anxiety | 9 | 56 | 21 | 83 |
| | 10.1% | 9.4% | 8.4% | |
| Bipolar | 15 | 30 | 21 | 66 |
| | 16.9% | 5.1% | 8.4% | |
| Column | 89 | 594 | 251 | 934 |
| Total | | | | |

The association was tested between pre-existing affective disorder and various genetic markers including APOE (APOE2, 3, 4), Hfe (C282Y, H63D, S65D), and methylene tetrohydrofolate reductase (MTHFR) alleles (A,V), and AAT phenotypes. Only AAT showed significant association, as demonstrated in table below. Common AAT ‘deficiency’ polymorphisms S, Z, and rarer AAT alleles were encountered significantly more frequently in persons with pre-existing anxiety disorder or bipolar disorder (Chi squared=140.9, p<0.00001). The possible relationship of this association with testing for or presence of particular APOE genotypes was explored and no relation was found for persons with or without APOE genotyping, of for persons with 0, 1 or 2 APOE4 alleles. Likewise, no relationship was observed with subgroups defined by C-reactive protein quartiles, ceruloplasmin quartiles, ferritin quartiles, Hfe genotypes, or MTHFR genotypes. A separate association exists for APOE2 carriers (APOE2/2 and 2/3) with increased frequency of S and Z polymorphism in persons with early presentation and white matter disease (ca. 25-30%, p=0.007).

Frequency Table for AAT polymorphisms and affective disorders

| AAT | | | | | |
|---|---|---|---|---|---|
| M . . . | None | Depression | Anxiety | Bipolar | Totals |
| MM | 616 | 94 | 59 | 34 | 803 |
| | 90.3% | 92.2% | 71.1% | 51.5% | 86.1% |
| MS | 49 | 6 | 16 | 15 | 86 |
| | 7.2% | 5.9% | 19.3% | 22.7% | 9.2% |
| MZ | 9 | 2 | 6 | 14 | 31 |
| | 1.3% | 2.0% | 7.2% | 21.2% | 3.3% |
| FM | 1 | 0 | 0 | 0 | 1 |
| IM | 5 | 0 | 1 | 1 | 7 |
| GM | 1 | 0 | 0 | 1 | 2 |
| PM | 1 | 0 | 1 | 0 | 2 |
| VM | 0 | 0 | 0 | 1 | 1 |
| Total | 8 | 0 | 2 | 3 | 13 |
| rare | 1.2% | 0% | 1.4% | 4.6% | 1.4% |
| Total | 682 | 102 | 83 | 66 | 933 |

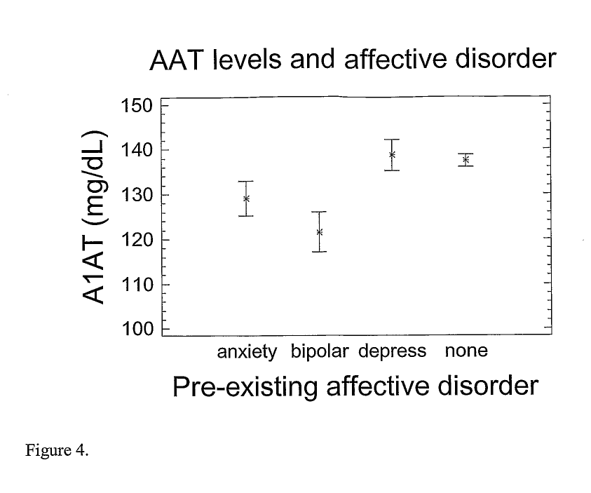

The above table demonstrates the significant association of AAT polymorphisms S and Z with the patient groups with pre-existing bipolar disorder and anxiety disorder. In addition, we asked the question whether AAT levels would also differentiate between the above categories. AAT levels are indeed significantly lower in persons with bipolar disorder and anxiety disorder when adjusted for gender and age, but not AAT polymorphism as demonstrated in MANOVA diagram (FIG. 4). This relationship is dependent on significant AAT polymorphism effect on AAT levels and is not observed for persons with MM subtypes, or in subcategory of persons with MS phenotype. However, in MZ persons, a significantly lower level of AAT was associated with bipolar disorder and anxiety disorder.

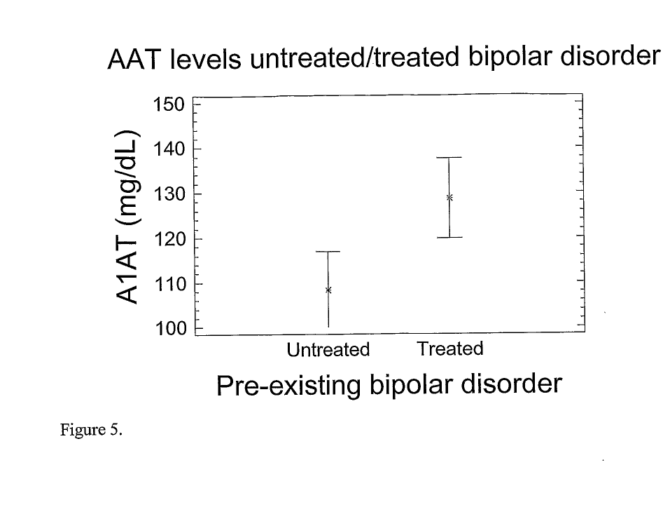

Bipolar disorder (a spectrum) was divided into: clinical diagnosis without drug therapy, clinical diagnosis with active therapy (lithium, mood stabilizers, etc), ADD/ADHD presentations, and those persons with mention of schizoaffective disorder, eating disorder, explosive disorder. In all above categories, elevated and significant proportion of non-MM AAT phenotypes was found. We found that a significant difference in AAT levels between untreated and treated persons with bipolar disorder spectrum. Increased AAT levels were observed in treated persons (lithium and/or valproic acid) and might be due to an endophenotype of bipolar disorder and/or drug effect of bipolar medications. See FIG. 5.

Because of the striking association of certain pre-existing affective disorders with A1AT phenotype of MZ, we explored the distribution of primary cognitive disorder diagnoses in this group (presented below). Of note is the presence of 3 cases of atypical demyelinating disease presenting as cognitive disorder with few motor signs or symptoms) and relatively equal numbers of AD/vascular dementia and MCI-amnesic and MCI-vascular.

Diagnostic Categories for 31 Persons with AAT MZ
Normal (n=1)
CIND (cognitive impairment, no dementia) (n=6)
Mild cognitive impairment (MCI)—amnesic (n=7)
Mild cognitive impairment (MCI)—vascular (n=5)
Alzheimer Disease (n=2)
Alzheimer Disease/vascular dementia (n=2)
Demyelinating or dysmyelinating disease (n=3)
Primary progressive aphasia (n=1)
Frontotemporal dementia (n=3)
Dementia of unknown etiology (n=1)
14 of these 31 patients were genotyped for APOE. The allele frequency for APOE4 was increased at 0.289 without significant difference from MM at 0.343 (compare to population expected ca. 0.15). There was no significant difference between AAT phenotype MZ and diagnostic families (CIND/normal, AD-related, non-AD) or with individual primary cognitive diagnoses [other than secondary diagnoses of pre-existing affective disorder as discussed above]. The exception was presentations with dysmyelinating or demyelinating reactions after multiple immunizations as discussed below.

Because of the striking association of certain pre-existing affective disorders with AAT phenotype of MS, we explored the distribution of primary cognitive disorder diagnoses in this group (presented below). Of note is the absence of demyelinating disease and relatively greater numbers of AD compared to vascular dementia and of MCI-amnesic compared to MCI-vascular.

Diagnostic Categories for 86 Persons with AAT MS
Normal (n=2)
CIND (n=7)
Mild cognitive impairment (MCI)—amnesic (n=25)
Mild cognitive impairment (MCI)—vascular (n=4)
Alzheimer Disease (n=18)
Alzheimer Disease/vascular dementia (n=11)
Demyelinating disease (n=0)
Primary progressive aphasia (n=4)
Frontotemporal dementia (n=7)
Dementia of unknown etiology (n=2)
Others not Found with MZ:
Alzheimer Disease (Downs syndrome) (n=1)
Alzheimer Disease—Parkinsonism (n=1)
Vascular dementia (n=2)
Corticobasal degeneration (n=1)
Cerebral degeneration with ataxia (n=1)

60 of the 86 persons were genotyped for APOE. The allele frequency for APOE4 was increased at 0.35 without significant difference from MZ at 0.289 or MM at 0.343 (compare to population expected ca. 0.15). There was no significant difference between AAT phenotype MS and diagnostic families (CIND/normal, AD-related, non-AD) nor with individual diagnoses [other than secondary diagnoses of pre-existing affective disorder as discussed above].

The above results support the identification of AAT deficiency polymorphisms and/or AAT deficiency (whatever the genetic and/or environmental causes) as a mixed genetic-environmental vulnerability factor in affective disorders such as bipolar disorders, anxiety disorders, and ADD-ADHD spectrum.

In a number of index probands cases, we were able to establish pedigrees:

### Example 1

Proband—Father, age 48: MZ-associated bipolar disorder (clinical, compensated) with white matter disease s/p multiple immunizations; Mother: unaffected; 1 offspring, age 17 with MZ-associated attention deficit disorder-ADHD s/p mild head injury.

### Example 2

Father, age 45: death from suicide, genotype unknown; Proband—Mother, age 72: MS-associated dementia syndrome c/w AD-vascular; child, age 42: frequent childhood ear infections, ADD, genotype MS; all three offspring of this child (grandchildren) with early onset ADD, genotype pending; another child of probands with bipolar disorder, genotype pending.

### Example 3

Father, age 57, MM genotype; Proband—Mother, age 62, SS genotype, frequent childhood ear infections, chronic bronchitis, anxiety/mild bipolar disorder; Maternal uncle, died age 58, —S genotype, ADD-childhood; 3 offspring of proband: 1 child, age 33, MS genotype, ADD—anxiety disorder; 1 child, age 31, MS genotype, anxiety disorder; 1 child, age 26, MS genotype, frequent childhood ear infections, anxiety disorder.

### Example 4

Family with four siblings: Probands—two sisters with bipolar disorder (MZ phenotype), two siblings without bipolar/anxiety—normal MM phenotypes. One offspring of bipolar person—MZ phenotype—asymptomatic.

This supports that genetic risk for anxiety disorders, bipolar spectrum, ADD-ADHD may segregate in certain families with A1AT deficiency polymorphisms and manifest in different persons of the same pedigree with different affective disorders. Typical family with MS or MZ persons in pedigree may also have increase or noticeable reactive airway disease, asthma, chronic bronchitis, COPD, liver disease, reactive hypoglycemia, childhood ear infections, etc. which have been associated with S and Z polymorphisms. Presumptive mechanism is “two-hit” in many cases with environmental factor interacting in utero, early development or later. This two-hit genetic-environmental model is totally consonant with concepts of AAT polymorphisms and liver disease (hepatitis C, alcohol use, etc) and pulmonary disease (smoking, chemical exposure, etc). Effects of AAT on iron metabolism, macrophage/microglial activation and transferrin receptor would support possible effects at different developmental time points and ages on radial glial cell—astrocyte transition, oligodendrogenesis and myelination, and neural development, as well as response to multiple immunizations (ADEM), CNS injury and neurodegeneration/injury repair.

Abnormal white matter on brain MRI or CT scans was commonly observed in persons with AAT ‘deficiency’ alleles with so-called patterns of T2-abnormalities characterized as ‘small vessel disease’ and similar neuroradiological appellations. In particular, persons with Z allele had significantly more abnormality of subcortical white matter.

Frequency Table of brain imaging [no abnormality,
small vessel disease (svd), severe svd or white matter
disease, or infarcts] vs. AAT phenotype

| AAT | | | | | |
|---|---|---|---|---|---|
| M . . . | No abnorm | Svd | Severe svd | Infarcts | Total |
| M | 157 | 111 | 47 | 15 | 330 |
| | 47.6% | 33.6% | 14.2% | 4.6% | |
| S | 22 | 16 | 5 | 0 | 43 |
| | 51.2% | 37.2% | 11.6% | 0% | |
| Z | 4 | 5 | 5 | 0 | 14 |
| | 28.6% | 35.7% | 35.7% | 0% | |
| Totals | 183 | 132 | 57 | 15 | 387 |

The proportion of cases with severe small vessel disease (bolded) was increased in persons with MZ phenotype (p=0.06). 2 out of 7 cases presenting with ‘multiple sclerosis’ were MZ carriers and, in that instance, both cases were veterans with history of acute disseminated encephalomyelitis (ADEM) after multiple immunizations for deployment (p<0.007).

### Example 3

### AAT and Age at Onset

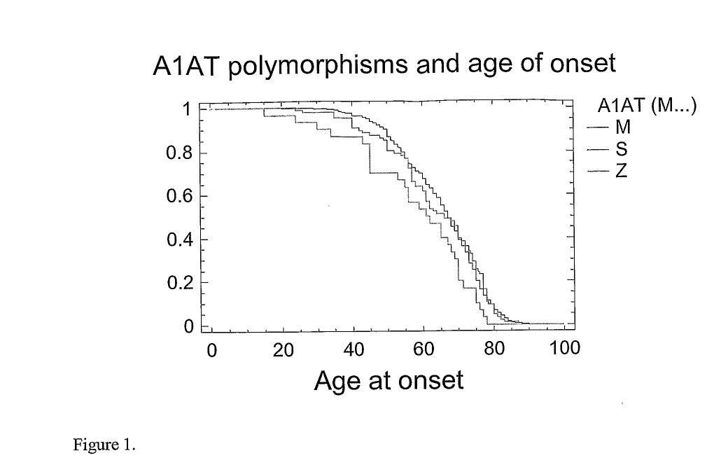

Fourteen of the 933 patients lacked age of onset data (normal/CIND) and 13 had rare AAT phenotypes. 906 remaining patients were analyzed with Kaplan-Meier curve. There was a statistically significantly earlier age of onset (average was 5 years) earlier in MZ (p=0.02) compared to MM and MS phenotypes (FIG. 1).

AAT polymorphisms affect age of onset or presentation for persons at risk or with cognitive dysfunction or dementia. This effect is most marked for AAT Z heterozygotes among the common ‘deficiency’ heterozygote states. See FIG. 1.

Age of onset for cognitive symptoms according to AAT polymorphisms with M signifying various M, M1, M2 combinations, S signifying MS heterozygotes and Z signifying MZ heterozygotes. Difference is significantly earlier, p=0.02 (Log rank), for MZ versus other phenotypes.

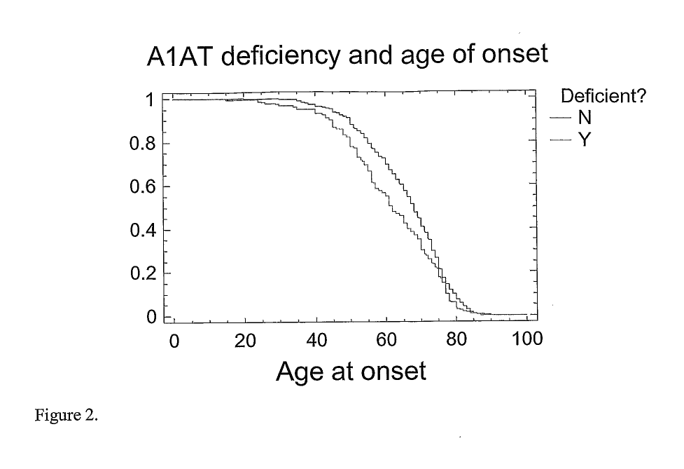

This effect on earlier age of onset is affected by ‘deficiency’ polymorphisms, but is also seen when only the level of AAT expression is considered. FIG. 2 shows age of onset for persons with deficient AAT levels compared to persons with normal levels at presentation.

Age of onset curves for set of persons with AAT polymorphisms associated with deficiency (MS, MZ) regardless of AAT level combined with normal phenotypes (M subclasses) with low level (<110 mg/dL). Earlier onset is significant (chi squared=12.6, p=0.0004, Log rank). Average (50^{th} percentile) age of onset is 6 years earlier for persons with AAT deficiency. When comparison is restricted to persons with normal M polymorphisms (M, M1, M2, . . . ) combinations and low AAT levels, effect remains with average 8 year earlier onset (chi-squared=14.7, p=0.0001, Log rank). The effect is most pronounced for female gender. In non-AD group, average AOO was early without significant difference in three AAT phenotypes (⅔ of MZ phenotypes are in non-AD.

A subset of persons genotyped for APOE (n=571) was analyzed for effects on average age of onset of APOE polymorphisms and demonstrated expected effect of APOE4 (2 E4 alleles, AOO=66 years; 1 E4 allele, 70 years; no E4 allele, 71 years; chi squared=20.8, p=0.00002, Log rank). Another subset genotyped for Hfe C282y allele showed mild effects (2 C282Y alleles, AOO=70 years; 1 C282Y allele, AAO=68 years; no C282Y alleles, AOO=62 years; chi squared=6.0, p=0.05, Log rank).

Taking APOE44 homozygote individuals with clinical diagnosis of MCI or AD as group with high likelihood of AD pathology, we examined effect of AAT deficiency, AAT, Hfe, and MTHFR polymorphisms on age of onset. There was no significant effect of AAT phenotypes, AAT levels, or MTHFR polymorphisms on age of onset. Presence of C282Y polymorphism did delay average age of onset in 73 persons with APOE4/4 AD and Hfe genotyping by ca. 5 years (no C282Y mutation, AOO=64 years (25^{th}, 75^{th} percentiles—58, 71) compared to presence of C282Y mutation, AOO=69 years (25^{th}, 75^{th} percentiles—67, 74.6). with chi-squared=5.23, p=0.02. Since ca. 65 years of age is usual average AOO for APOE4/4 individuals, this would represent an apparent delaying of onset of APOE4/4 homozygote AD by presence of Hfe C282Y polymorphism.

### Example 4

### Interactions of Iron Metabolism and AAT Phenotypes

After adjusting only for age, gender and C282Y/H63D mutations in Hfe gene, AAT phenotypes do not influence appreciably iron indices such as serum iron, transferrin, ferritin, transferrin index. (see table below). However, when inflammation as measured by c-reactive protein (Crp) is accounted for, there are significant differences in persons with deficiency polymorphisms (next section).

AAT and relationship to iron metabolism adjusted for age, gender

| AAT | Gender | AAT | Iron | Transferr | Ferritin | Crp |
|---|---|---|---|---|---|---|
| MM | Male | 135 ± 21 | 82 ± 31 | 249 ± 38 | 155 ± 161 | 0.31 ± .54 |
| | (340) | | | | | |
| MM | Female | 144 ± 25 | 73 ± 29 | 263 ± 49 | 107 ± 296 | 0.45 ± .99 |
| | (456) | | | | | |
| MS | Male | 122 ± 23 | 76 ± 30 | 241 ± 39 | 141 ± 97 | 0.49 ± .96 |
| | (44) | | | | | |
| MS | Female | 121 ± 22 | 77 ± 31 | 265 ± 45 | 95 ± 76 | 0.30 ± .34 |
| | (41) | | | | | |
| MZ | Male | 77 ± 13 | 83 ± 25 | 253 ± 40 | 152 ± 152 | 0.22 ± .24 |
| | (15) | | | | | |
| MZ | Female | 94 ± 20 | 79 ± 27 | 247 ± 62 | 116 ± 139 | 0.51 ± 0.90 |
| | (14) | | | | | |

[AAT in mg/dL, iron in ug/dL, transferrin in mg/dL, ferritin in ng/ml, crp in mg/dL]

Iron indices with respect to AAT polymorphism adjusted for age, gender, AAT level (1^{st} column represents values for crp<0.40 mg/dL (low inflammation), 2^{nd} column for each variable represents values for crp>−0.40 mg/dL (high inflammation). These values show that AAT values are increased in high inflammation group, but less so in MZ phenotype. However, ferritin values are significantly increased in MZ phenotype with high inflammation (bolded right lower corner), proportionately much more than MM and MS groups.

AAT and relationship to iron metabolism adjusted for age, gender, AAT, c-reactive protein
and divided into low inflammation (left column) and high inflammation (bold)

| AAT | Gender | AAT | AAT | Iron | Iron | Transferr | Transferr | Ferritin | Ferritin |
|---|---|---|---|---|---|---|---|---|---|
| MM | Male | 130 ± 1 | 152 ± 4 | 84 ± 3 | 84 ± 9 | 250 ± 3 | 253 ± 17 | 156 ± 11 | 112 ± 44 |
| | (340) | (240) | (51) | | | | | | |
| MM | Female | 140 ± 1 | 151 ± 3 | 77 ± 2 | 64 ± 3 | 263 ± 3 | 262 ± 5 | 91 ± 5 | 108 ± 12 |
| | (456) | (281) | (101) | | | | | | |
| MS | Male | 114 ± 3 | 136 ± 9 | 76 ± 8 | 92 ± 13 | 258 ± 11 | 233 ± 24 | 148 ± 32 | 96 ± 23 |
| | (44) | (25) | (9) | | | | | | |
| MS | Female | 123 ± 4 | 131 ± 8 | 76 ± 6 | 65 ± 7 | 258 ± 10 | 283 ± 15 | 90 ± 16 | 118 ± 34 |
| | (41) | (26) | (12) | | | | | | |
| MZ | Both | 86 ± 7 | 93 ± 13 | 78 ± 7 | 83 ± 10 | 279 ± 20 | 261 ± 11 | 93 ± 23) | 274 ± 49 |
| | (29) | (18) | | | | | | | |

Copper indices with respect to AAT polymorphism adjusted for age, gender, AAT level, and crp level: 1^{st} column represents values for crp<0.40 mg/dL (low inflammation), 2^{nd} column for each variable represents values for crp>=0.40 mg/dL (high inflammation).

AAT and relationship to copper metabolism adjusted for age, gender, AAT, c-reactive protein

| AAT | Copper | Copper | Zinc | Zinc | FreeCu | FreeCu | Freecu % | FreeCu % | Cp | Cp |
|---|---|---|---|---|---|---|---|---|---|---|
| MM | 116 ± 1 | 132 ± 4 | 84 ± 1 | 80 ± 2 | 35 ± 1 | 40 ± 2 | 29.4 ± 0.4 | 29.7 ± 0.9 | 27.1 ± 0.3 | 31.2 ± 0.8 |
| (426) | (288) | (80) | | | | | | | | |
| MS | 124 ± 4 | 139 ± 10 | 85 ± 3 | 85 ± 5 | 40 ± 2 | 46 ± 6 | 30.9 ± 1.2 | 32.6 ± 2.4 | 28.1 ± 0.9 | 33.1 ± 2.2 |
| (54) | (25) | (9) | | | | | | | | |
| MZ | 131 ± 7 | 185 ± 15 | 79 ± 5 | 96 ± 7 | 40 ± 4 | 50 ± 9 | 29.9 ± 2.3 | 24.5 ± 3.8 | 30.0 ± 1.7 | 40.8 ± 3.3 |
| (21) | (9) | (3) | | | | | | | | |

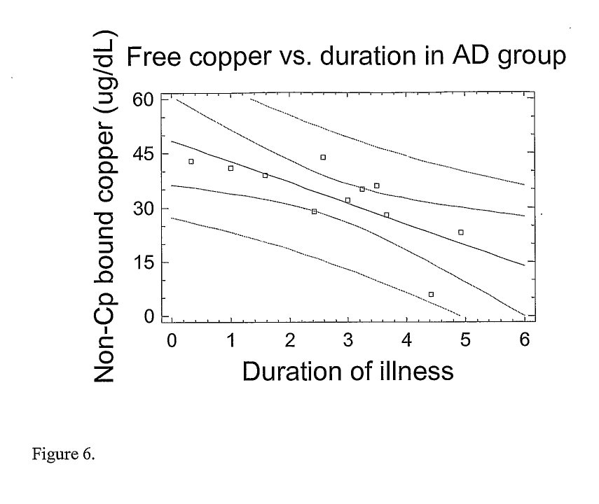

Above shows that inflammatory “drive” as measured by copper/zinc ratios is considerably and significantly increased for MS and MZ individuals compared to MM (copper/zinc ratios, MM: 1.43±0.02—normal; MS: 1.51±0.07; MZ: 1.71±0.12) even in low inflammatory conditions (crp <0.40 mg/dL) and this AAT-dependent relationship is enhanced in high inflammatory conditions (crp>=0.40 mg/dL) (copper/zinc ratios, MM: 1.62±0.19, MS: 1.72±1.07, MZ: 1.99±0.03). Instead of average 16% increase in serum copper, MZ individuals raise serum copper an average of 50%. Thus, their free copper is increased, but disproportionately less in relation to total copper with less percentage free copper. This supports concept that these individuals have two problems: abnormal iron stores and circulating iron, higher ferroxidase (ceruloplasmin), more circulating non-ceruloplasmin bound copper (vital to nervous system and end-organ copper transport, but potentially oxidative to proteins and cells) and less tissue copper stores through ‘chronic’ copper wasting (8). This relationship is time-dependent in MZ individuals and in persons with C282Y carrier state (r2=−0.55, p=0.009). FIG. 6.

The relationship between ‘free’ or non-ceruloplasmin bound copper and AAT expression levels is AAT polymorphism and gender dependent. In male gender, AAT genotypes MS and MZ or MM genotypes with low levels do not correlate with free copper release (in fact, they are negatively related, but not significant). In female gender, AAT is positively correlated with ‘free’ copper levels. In MM AAT phenotype of both genders with normal AAT levels, there is a linear relationship between ‘free’ copper and AAT levels.

The effect of AAT levels on age of onset is affected by other genetic polymorphisms including those affecting iron metabolism:

1) Hemochromatosis gene: C282Y (+/−) or HET carriers show no effect of low AAT levels on age of onset
2) Hemochromatosis gene: C282Y normal or wild type (−/−)—significant lowering of age of onset with low AAT levels (chi squared=12.6, p=0.0003) of average 7 years earlier age of onset.
3) Apolipoprotein E gene (APOE): APOE44 homozygotes—no effect of low AAT levels on age of onset [perhaps dominant effect can not be overcome].

The effect of AAT levels on age of onset is affected by peripheral biochemical markers including those affecting iron metabolism:

1) Interaction of AAT phenotype MS with highest (4^{th}) transferrin quartile:

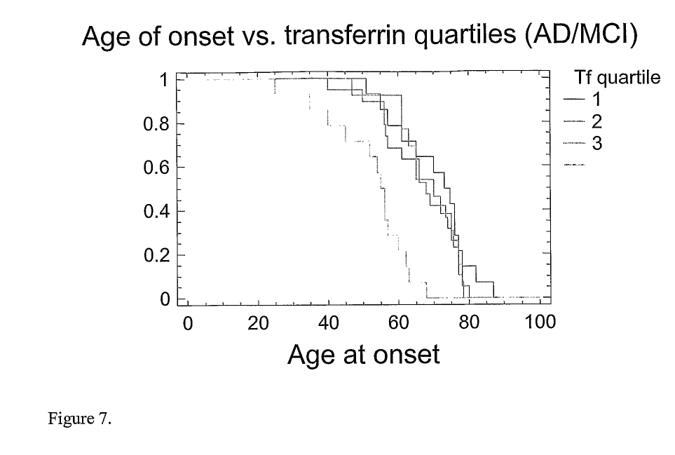

There is significant lowering of age of onset (n=53 persons, chi squared=21.0, p=0.0001) by average of 19 years earlier with low AAT levels in group in 4^{th} or highest quartile of transferrin levels. There is no interaction with lowest transferrin quartiles (1^{st}, 2^{nd} and 3^{rd}). Transferrin quartiles were determined for all persons with AAT levels: 1^{st} up to—226 mg/dL; 2^{nd}—226-250 mg/dL; 3^{rd}—250-280 mg/dL; 4^{th}—280 mg/dL and higher). FIG. 7.

This relationship between transferrin quartiles and age of onset is confined to AD and related dementias and is not observed for diagnosis group 3 (non-AD). For those AD/MCI persons with low AAT level, effect is still significant with change in age of onset of ca. 17 years (n=123 persons, chi squared=24.6, p=0.00002, Log rank). For entire group of AD/MCI persons, effect is nevertheless observed more modestly with change in age of onset of ca. 3 years (n=755 persons, chi squared=17.1, p=0.0007). This relationship of earlier age of onset is not observed for quartiles of other acute phase reactants such as ferritin, ceruloplasmin, c-reactive protein or copper/zinc ratio and thus is specific to transferrin.

2) Effect is observed in persons with deficient or low thiamine (vitamin B1) levels, but is lessened in persons presenting with normal thiamine levels.
3) Effect is unrelated to LDL or HDL levels.
4) Effect is not observed in diabetics. The effect of AAT levels on age of onset is abrogated by glucose intolerance/presence of diabetes and is not observed in diabetics. Note that AAT levels are higher on average by 10 mg/dL in diabetics
5) Copper/ceruloplasmin ratio for AAT deficiency vs. Hfe genotypes

A significant increase in ratio of copper (ug/dL) per ceruloplasmin (mg/dL) was observed for AAT deficiency in iron overload (HET), but actually a decrease in wild type persons. Recall that effect of AAT deficiency on age of onset was confined to wild type hemochromatosis cases.

Copper/ceruloplasmin ratio for Hfe
Genotypes and A1AT deficiency

| AAT | | | | |
|---|---|---|---|---|
| Def | HET | Het | Hom | Wildtype |
| No | 4.20 (26) | 4.29 (61) | 4.23 (10) | 4.33 (138) |
| Yes | 4.52 (12) | 4.47 (18) | 3.76 (2) | 4.16 (62) |
| | P = .04 | P = .08 | P = .11 | P = .02 |

[HET = C282Y+/−, H63D−/−; Het = C282Y−/−, H63D+/−; Hom = C282Y−/−, H63D+/+; wild type = C282Y−/−, H63D−/−].

6) Copper/zinc ratio for AAT deficiency vs. Hfe genotypes

A significant decrease in copper/zinc ratio was observed for Hfe wildtype persons (great majority, ca 85-90%) with AAT deficiency by levels or ‘deficiency’ phenotypes S and Z.

Copper/ceruloplasmin ratio for Hfe
Genotypes and A1AT deficiency

| AAT | | | | |
|---|---|---|---|---|
| def | HET | Het | Hom | Wildtype |
| No | 1.40 (26) | 1.44 (60) | 1.55 (10) | 1.51 (136) |
| Yes | 1.52 (11) | 1.44 (17) | 1.46 (2) | 1.37 (62) |
| | P = NS | P = NS | P = NS | P = .02 |

[HET = C282Y+/−, H63D−/−; Het = C282Y−/−, H63D+/−; Hom = C282Y−/−, H63D+/+; wildtype = C282Y−/−, H63D−/−].

### Example 5

### AAT levels and Progression Rate in AD

We examined the relationship of change in mini-mental status examination scores (MMSE) with regard to different inflammatory markers including AAT levels. FIG. 2 shows that there is a relationship of higher AAT levels with greater stability in test-retest scores over time periods of greater than 1 year for patients with MCI/AD. We tested a number of variables relevant to progression rate to assist in model selection.

### Demographic Variables:

1) Age of onset (aoo): younger onset cases progress on average more rapidly than older onset cases.
2) Gender: females progress on average more rapidly than males.

### Genetic Variables:

1) APOE4 alleles: there was a significant graded effect of 0, 1 or 2 APOE4 alleles on MMSE change scores as described in the literature.
2) Hemochromatosis gene polymorphisms: persons with 1 or 2 C282Y alleles were significantly more stable than persons with wildtype [not found in literature].

### Biochemical Variables:

1) Significant continuous variables with positive correlation for less progression (i.e., stability) in MMSE scores included AAT levels (p=0.01), serum copper (p=0.04), copper/zinc ratio (p=0.02), ceruloplasmin (p=0.01), tendency for HDL (p=0.14), but not free copper, copper/ceruloplasmin ratio, c-reactive protein, fibrinogen, LDL cholesterol, serum zinc. Continuous variables with tendency for negative correlation for less progression included transferrin index (p=0.09), serum iron (p=0.09), serum ferritin (p=0.09).
2) This analysis supports the use of AAT as a potent predictor of progression rate; the more enhanced the AAT component of the inflammatory reaction (macrophage activation) is compared to general indices of inflammation (e.g., c-reactive protein) the more stable the patient. This is consistent with the concept that failed inflammatory reactions underlie some of the AD pathology and that AD pathology can be “removed” if sufficient normal immunological and inflammatory mechanisms are brought to bear.

### General Linear Equation:

Given above theoretical approach, literature and discoveries reported in this invention, a general linear expression was utilized to check for ability to account for observed variance in progression rates.

Variables were age of onset, AAT level, and c-reactive protein as continuous variables and gender, APOE4 alleles, and C282Y allele status (present in 1-2 copies or absent) as categorical variables for cases with initial MMSE>23, interval to retest of at least one year, and diagnosis of MCI/AD. 59 cases were suitable.

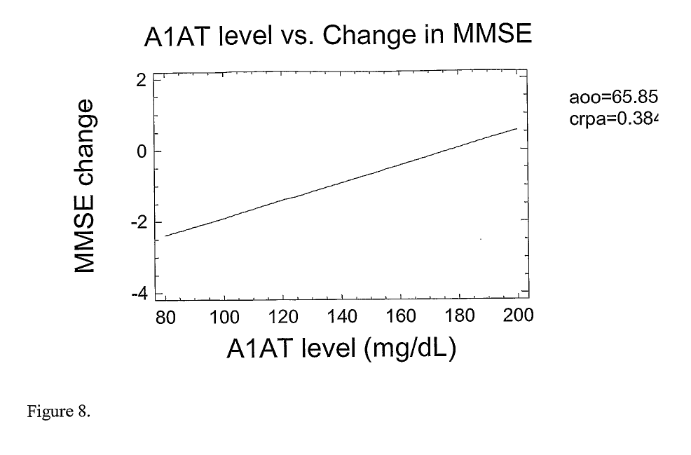

Results were model accounting for 39% of variance (sum of squares). Model utilized age of onset (p=0.012, F=11.9), AAT level (p=0.01, F=6.3), c-reactive protein level (p=0.01, F=6.4). Categorical variables were just beyond significance, but improved overall model (gender, p=0.17; presence of C282Y alleles, p=0.28; and APOE4 alleles (0, 1, 2) nearly significant, p=0.07). FIG. 8.

The model and coefficients from above were specifically:

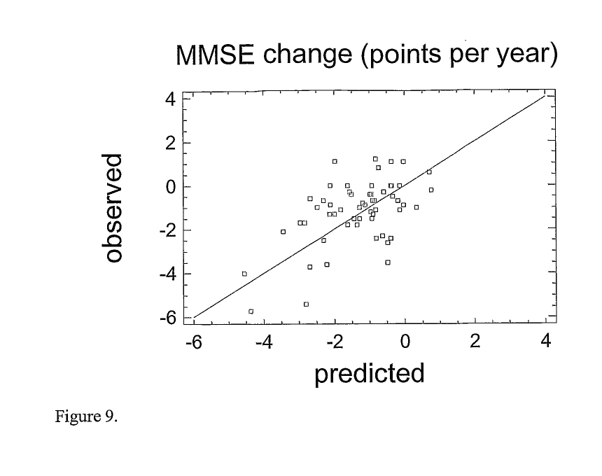

MMSE (change per year)=−10.31+(−0.32)*(1 for female, −1 for male)+(−0.287)*(1 for wild type C282Y, −1 for C282Y carrier or homozygote, 0 otherwise)+(0.524)*(1 for no APOE4 alleles, −1 for two APOE4 alleles, 0 otherwise)+(0.187)*(1 for one APOE4 alleles, −1 for two APOE4 alleles, 0 otherwise)+(0.095)*(AAT)+(−0.733)*(CRP). The predicted vs. observed values from this model are presented in FIG. 9.

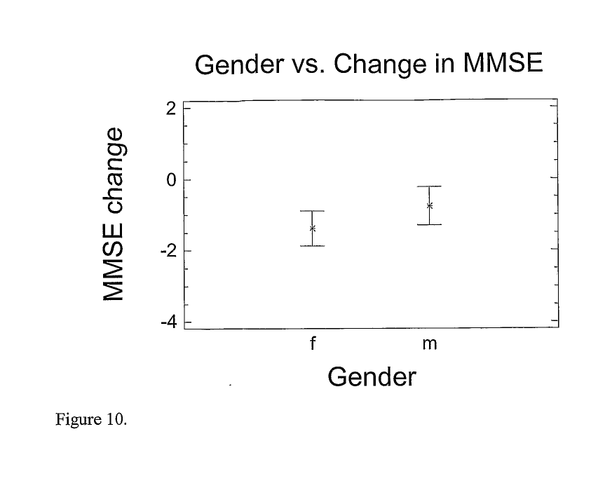

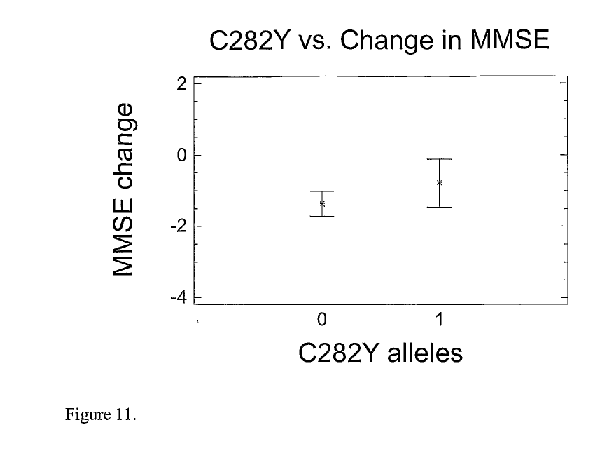

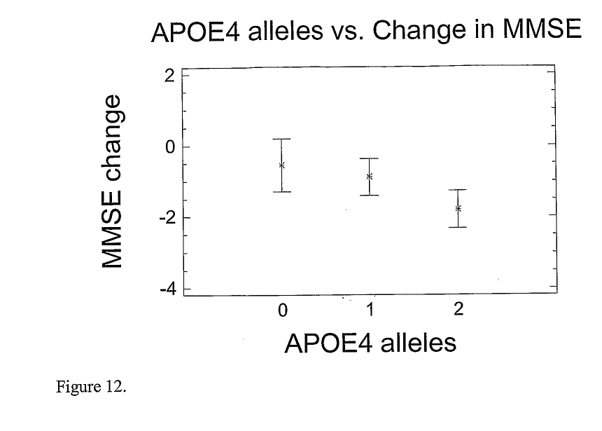

FIGS. 10-12 present the MMSE change for categorical variables of gender, APOE4 and hemochromatosis alleles.

### Logistic Regression Model and Discriminant Analysis

In order to provide an estimate of clinical utility, the cases were categorized as stable (MMSE score <=1 units change per year) vs. progressive (MMSE score >1 units change per year). These unitary values for stability were then used in a logistic regression model to examine variables and coefficients that might help predict likelihood of stability over time. Persons with initial MMSE scores of >23 (so called MCI range or early AD) with interval from test to retest of greater than one year were chosen to illustrate model.

For cases with APOE4/4 homozygote status (n=30), the above logistic regression model utilized age of onset (AOO) (yrs), AAT level (mg/dL) and c-reactive protein (CRP) (mg/dL) to yield an equation (2^{nd} order), representing best fit to observed stability:

Eta=−6.34+0.000652*AAT*AOO+0.2086*CRP*AAT−58.54*CRPÂ2

Then, predicted or fitted values=exp(eta)/(1+exp(eta)).

Discriminant analysis of observed stability values vs. range of values from 0-1 produced by the above equation shows 83.3% of cases correctly classified (see table below).

Classification Table for cases homozygous for E44

| | Predicted unstable | Predicted stable |
|---|---|---|
| Observed | 10 | 1 |
| unstable | 90.9% | 9.1% |
| Observed | 4 | 15 |
| stable | 21.1% | 79.0% |

When applied to a larger series of cases of all genotypes (n=38) whose ultimate diagnosis was AD, the above equation results in 86.8% of cases correctly classified (see table below). Breakpoint for discrimination was all values above 0.59 assigned to stable category.

| | Predicted unstable | Predicted stable |
|---|---|---|
| Observed | 15 | 4 |
| unstable | 79.0% | 21.1% |
| Observed | 1 | 18 |
| stable | 5.3% | 94.7% |

This classification table demonstrates and can be used to calculate the following measures of clinical utility (chi squared=18.2, p=0.00001):

### Sensitivity=94.7%: Specificity=78.9%

### Relative Risk=13.1 (2.7-256): Odds Ratio=67.5 (5.8-1800)

Positive predictive value=81.8%: Negative predictive value=93.8%

Fitting the observed stability using other variables than age of onset, AAT and CRP levels did not result in significant results when the following were singly examined and in combination with AOO: ceruloplasmin, copper/zinc ratio, copper, CRP alone.

Approach is either for linear equation as described above using gender, age of onset, AAT, and CRP to predict MMSE change at given interval or as MMSE change per year estimate or for logistic regression equation using above variables and approach. Method is proposed so that larger numbers of persons in MCI/AD range (MMSE>22-23) could be analyzed by this approach to yield coefficients more suited for particular patient groups. These results predict for large groups that accounting for AAT phenotype, Hfe genotype, and APOE genotype would improve model (see above). For the logistic regression approach, the MMSE change per year considered “stable” was taken as decrease of 1 point per year or less based on literature, but alternative “cut-points” could be chosen.

Utility for basic and clinical research and for clinical practice of the method and approach would be to triage patients by predicted MMSE score as covariate in treatment outcomes or to bin patients by unstable and stable categories. For clinicians, the ability to grade patients with AD dementia into stable and unstable would be beneficial for gauging patient follow-up, aggressivity of evaluation and treatment, and prognosis for patient and family.

### Example 6

### Comment/Summary

MS and MZ phenotypes of AAT are slightly increased in our clinic-based series (13-15%), possibly due to selection bias given range reported in U.S. populations (1). Normal/CIND persons also showed this slightly higher frequency. MS and MZ polymorphisms are rare in African-Americans (1 out of 21 in this series) and other polymorphisms need to be examined (15). Subset with previously diagnosed affective disorder (anxiety or bipolar disorder spectrum) had a significantly higher proportion of MS and MZ phenotypes (29% of persons with anxiety disorders and 49% of persons with bipolar disorder, p<0.00001). Indeed, once this subset of persons with anxiety or bipolar disorder is removed, the frequency of S and Z polymorphisms in the remaining population in our series is similar to expected frequency of 9% (1). This supports strong association of AAT with anxiety and bipolar disorder and/or with cognitive decline in affective disorders. There is linkage for bipolar disorders on chromosome 14q (16). Another subset with high MS and MZ prevalence (20-30%, p<0.007) were persons with APOE2/2 or APOE2/3 genotype. AAT may represent a modifier for normally “protective” APOE2/3 genotype (9, 17). We have clinical evidence for presentation of attention-deficit hyperactivity disorders in both younger and older patients carrying A1AT polymorphisms S and Z. This includes observation that there is segregation and phenotypic variation of ADD/ADHD, anxiety disorder and bipolar disorder with AAT deficiency polymorphisms in many family pedigrees. We find significantly greater subcortical white matter disease in some persons with Z alleles and possibly S alleles. This may interact with vasculopathic factors as well as acquired insults or 2^{nd} hits such as multiple immunizations, acute disseminated encephalomyelitis or other biological/chemical/toxic or immunological producing nervous system injury particularly to white matter or blood vessels. Based on literature on radial glial/astrocytic transitions in neurogenesis, on “pruning” of connections in nervous system during development, and on effects of altered iron/copper metabolism on oligodendrocytes and myelination, we propose that AAT polymorphisms may alter in selectively advantageous ways neural development (discernible through neuropsychological testing and/or imaging methodology), but also confer increased susceptibility to 2^{nd} hits or environmental factors during prenatal and postnatal development including immunization reactions, recursive effect of AAT effect on otitis media, fever, and other nervous system injuries as described above.

AAT levels are stable in a given person, but vary widely. Significantly low AAT levels (<110 mg/dL) are associated with an earlier AOO. Oxidized AAT was not assessed in this report and may be a factor for AAT effects on lipids and macrophages (18-21) and is reported as a specific measure of oxidative stress in AD (18). S and Z polymorphisms also relate to AOO. Persons with MZ present earlier than persons with MM and MS (Table 2). In non-AD, MM, MS and MZ survival curves ‘collapse’ together at an early AOO. In AD, AOO is significantly lower for MZ than MS and MM. Given the relation of AAT to iron metabolism (20), we separated MS curve by transferrin quartiles. There was significant earlier AOO MZ<MS<MM for highest transferrin quartile, but not for ferritin, AAT, ceruloplasmin or c-reactive protein quartiles. Factors affecting iron metabolism may interact with S and other deficiency polymorphisms on AOO in AD. These effects also held for all persons with low AAT levels with similar interaction with serum transferrin levels. The group of patients affected by transferrin modulation of AAT effects would minimally represent 20% of all AD/MCI patients.

AAT levels relate significantly to MMSE change and rate of progression for early AD (MMSE>23) with greater change at lower AAT levels (p<0.001). Effect was modulated by age, gender, and APOE4. Hfe C282Y carriers showed significant stability of MMSE scores compared to non-carriers (p<0.001). Transferrin levels are lower in C282Y carriers. Key variables are age of onset (AOO), gender, AAT level and measure of inflammatory drive such as c-reactive protein (CRP). Effective modeling can be accomplished with general linear equation to predict actual change in MMSE scores or with logistic regression (see examples above) to predict stability or non-stability with high sensitivity and specificity. The discovery predicts that this method can be adapted to larger sets of patients with same results and that this will be of great utility for determining progression rate, for use in pharmacological trials and for clinical evaluations, follow-up and treatment.

These results extend the list of disease susceptibilities associated with AAT polymorphisms and deficiency from lung/liver disease to ADHD/ADD spectrum, anxiety disorder, bipolar disorder spectrum, subcortical white matter disease, and to onset/progression rate for AD and related dementias. Clinically important subtypes or endophenotypes of AD and affective disorders are enriched in S and Z variants. Several possible mechanisms are supported by the known relationship of AAT to environmentally modulated pulmonary and liver disease and role of AAT in macrophage function, iron and lipid metabolism, and processing and deposition of aberrantly folded proteins (22-24). Possible gene-gene interactions of AAT, Hfe C282Y and APOE2 polymorphisms and likely role of environmental factors need more study to further interpret these results. Polymorphisms in either AAT (e.g., S, Z and other rare alleles) and/or Hfe (e.g., C282Y mutation), genes that affect iron and lipid metabolism and macrophage function, may be present in 20-30% of all patients presenting with cognitive disorders. Similar polymorphisms in A1AT and Hfe and relationships are inferred for Non-Western European populations. Our clinical series supports that iron overload and/or disorders of peripheral iron and copper metabolism, hepatosteatosis (non-alcoholic steatohepatosis—NASH or non-alcoholic fatty liver—NAFL), disordered glucose metabolism and thiamine deficiency commonly accompany these liver polymorphisms. Lower AAT levels are described as a risk factor for coronary artery disease progression (13), and low levels of AAT in brain as risk for progression in Huntington's disease.

Described actions of AAT include:

a) inhibition and complex formation with neutrophil elastase and cathepsin G: thus inhibiting elastase-mediated proteolytic breakdown of elastase (14, 19-21), and possibly intracellular and/or extracellular breakdown of ferritin and its contents (personal communication, A. Ghio, EPA, RTP, NC) This would result in free iron release and IRP/IRE activation and its downstream regulated targets such as APP)
b) interactions with fatty acids and bile acids on ‘gemfibrozil’ binding site (25)
c) interaction with SEC receptor mediating chemotactic effects (4, 26)
d) inhibition of angiogenesis (and tumor growth) (27)
e) transcriptional down-regulator of bile acid synthesis—C36 peptide with FTF (28)
f) target for metalloproteases (e.g, MMP-26) (29)
g) activation of macrophages/microglia (eg, TNF-alpha) through binding of C36 to class B scavenger receptor (CD36) and to LDL receptors and activation of reactive oxygen species (respiratory burst) (30)
h) modulation of endothelial NOS and effects of nitrosylation on AAT (31, 32, 33)
i) modulation of autophagy and mitochondrial dysfunction through effects on caspase and proteasome function and effect of fasting (12, 24)
h) mediator of iron metabolism switch for segregation vs. importation involved in erythropoiesis and in iron detoxification [inhibition of binding of TF to receptor, increase of intracellular ferritin] c/w changes of ‘anemia of chronic disease’. (14, 21)
i) complex formation with oxidized AAT and LDL for more rapid clearance (35)
j) modulation of transferrin receptor binding by transferrin, hemochromatosis protein and modulation of uptake, thereby affecting apoptosis in iron-sensitive cells (36-38)
k) anti-apoptotic factor for vascular smooth muscle cells (39)
l) inhibition of caspase-3 and -7 activation and inducible anti-apoptotic factor for inflammation, host defense (40)
m) inhibition of proteolytic processing of stalk region of transferrin receptor by neutrophil elastase and cathepsin G (41)
n) inhibition of diferric transferrin binding and uptake by skin fibroblasts (38)
o) stimulation of astrocyte proliferation (42)
p) presence and activity in AD lesions, as listed above (43)
q) inhibition of neutrophil elastase by AAT or by other compounds or enzymes such as Silvestat, SSR69071, active tamarind seed extracts or compounds, active alpha-ketooxadiazole compounds, SKALP/elafin or pre-elafin (trappin 2), or SLPI (55-65).

Models for AAT deficiency and by inference of this invention for anxiety, bipolar disorder and for disease onset/progression rates in dementia/AD include:
a) drosophila necrotic mutation model for “Z” variant (44, 45)
b) transgenic and targeted replacement animals containing human mutations of AAT and/or crosses and/or use of animals with AD-associated proteins (46)
c) appropriate neuronal, glial and/or macrophage cells in culture (current and future art)

### Targets for Treatment Include:

a) drugs altering conformational properties designed at 5 cavities of AAT (47, 48)
b) altering monoallelic to diallelic expression in relevant tissues or cells (49)
c) altering specific transcription factors such as HNF-1alpha and HNF4 (liver) (50, 51)
d) oxidation/nitrosylation groups on AAT and ratios to native ATT (see text)
e) C36 fragment (see text)
f) CD36 receptor and AAT modulation of amyloid beta (52)
g) Biochemical, pharmacological and genetic manipulation of AAT (see text)
h) Inhibition of transferrin receptor internalization and binding (common feature of AAT, hemochromatosis gene, and lithium) and/or ferritin induction so as to decrease free and non-bound iron (see text)
i) inhibition of neutrophil elastase or other protein or protease targets (see text)
j) effects on prevention of apoptosis (see text)
k) effects on LDL metabolism and receptors (53)
l) effects on proteasome degradation and glycosylation of AAT (54)
m) effects on complex formation with LDL, neutrophil elastase and other proteins

### Study Population and Clinical Evaluation

### Example 7

### Methods Used

1410 patients over the age of 20 years were examined one or more times between January, 2000 to June, 2005 in the Memory Disorders Clinic. Average age at onset (AOO) for 1383 patients with cognitive disorder was 65.1±12 years and age at presentation 68.6±12 years. 57.2% were female. Average follow-up for 648 patients with multiple visits was 1.47±1.47 years (range 0.3-9.5 y).

Primary diagnoses were based on practice guidelines (66, 67) and grouped: (1) normal and cognitively impaired non-demented (CIND); (2) amnesic mild cognitive impairment (aMCI), possible/probable AD, AD with Parkinsonism, AD with vascular disease, and Lewy Body dementia; and (3) other (non-amnesic) MCI, frontal lobe or frontotemporal dementia, primary progressive aphasia (PPA), vascular dementia, and other minor categories. aMCI represented persons with prominent memory complaints and missed recall on MMSE, and other MCI (non-amnesic and/or presumptive vascular) persons with behavioral and functional complaints and intact recall (66). Secondary neuropsychiatric diagnoses included previously diagnosed and treated major depression (10.8%, 126 persons), anxiety disorder (7.4%, 87 persons) and bipolar disorder (6.1%, 71 persons). Additional genetic testing and blood work was obtained based on guidelines for testing symptomatic patients and full work-up of vascular risk factors. Clinical indication for genetic and laboratory tests of liver function was related to common finding of abnormal iron indices, low or deficient plasma thiamine values (56% of 1210 persons tested had plasma thiamine <2.5 ng/ml [normal 0.5-9 ng/ml,SI conversion 10⁻⁹ g/L]), and/or palmar telangectasia.

### Biochemical Analysis

APOE (Athena Diagnostics) and Hfe genotyping (Duke) were performed using PCR, α-1-antitrypsin phenotyping by isoelectric focusing in polyacrylamide gels (Mayo Laboratories) and other routine tests through CLIA-approved laboratories of Duke University Medical Center on non-fasted specimens. Plasma thiamine was performed using HPLC (Cambridge Biochemical) and transferrin, ferritin, ceruloplasmin and fibrinogen using rate nephelometry (Duke). Serum iron, copper and zinc were performed by flame photometry. M, M1, M2 and other “M” phenotypes of AAT were labeled M.

### Statistical Analysis

IRB exemption was granted for retrospective analysis of clinical database on anonymized patient data. Analysis was by Chi-Squared test on cross-tabulation tables and by Paired T-test or ANOVA on single variable comparisons. Time of onset was assessed by Kaplan-Meier survival curves while differences between the curves were assessed by the Log rank statistic. Curve fitting for rate of progression analysis was performed using general linear equation, logistic regression packages, and discriminant analysis on commercial statistics platform (Statgraphics)

### Group Comparisons

Genetic tests were used for counseling and treatment selection, but not to change primary or secondary neuropsychiatric diagnoses. Rate of AAT testing increased from 2000-2004 (15% to ca. 100%). There was no significant difference for group with and without AAT testing except slight difference in presentation age.

For patients with AAT testing, APOE4 allele frequency was elevated in AD group (0.482) as expected (9), and was at background (0.160) in non-AD group (p<0.0001) with inverse relation present for APOE2 allele. Non-AD group was considered disease control. Hfe genotyping revealed allele frequency of 0.042-0.086 without significant inter-group difference. APOE4 allele frequency was elevated above background in groups with secondary neuropsychiatric diagnoses of depression, anxiety disorder or bipolar disorder (ca. 0.35).

AAT allele frequencies were equivalent by year of testing (p=0.96, data not shown). They are similar to reported values for US population (1) in normal and CIND group, and are not significantly different between AD and non-AD groups. Both AD and non-AD groups had expected lower MMSE at entry. There was greater proportion of females in AD compared to non-AD group. MCI diagnosis was common in both AD group (aMCI) and non-AD group, reflecting relatively mild and early stages of cognitive impairment typical of this clinic population.

TABLE 3

| | AAT Testing | | | | |
|---|---|---|---|---|---|
| | | | Normal/CIND | AD | Non-AD |
| Category | No AAT | AAT | Group | Group | Group |
| Number | 478 | 933 | 88 | 594 | 253 |
| Age of | 68.6 ± 12.1 | 68.1 ± 12.4** | 57.2 ± 12.6 | 72.3 ± 10.5 | 62.2 ± 12.0*** |
| Presentation, y | | | | | |
| Females(%) | 59% | 56% | 69% | 59% | 45%*** |
| Education(y) | 14.0 ± 3.4 | 13.9 ± 3.0 | 15.2 ± 2.9 | 13.6 ± 3.1 | 14.2 ± 2.8 |
| MMSE at entry | 23.7 ± 6.5 | 23.8 ± 6.0** | 29.2 ± 1.0 | 23.0 ± 5.8 | 24.3 ± 6.3** |

Paired T test: age of presentation, MMSE at entry; ANOVA: age of presentation, MMSE, education for three groups; chi squared analysis for gender.
*p < .05,
*p < .01,
**p < .001

### REFERENCES

1. Blanco I. Bastille E F. Rodriguez M C. Distribution of alpha1-antitrypsin PI S and PI Z frequencies in countries outside Europe: a meta-analysis. Clinical Genetics. 2001; 60(6):431-41.
3. Dahl M. Tybjaerg-Hansen A. Sillesen H. Jensen G. Steffensen R. Nordestgaard B G. Blood pressure, risk of ischemic cerebrovascular and ischemic heart disease, and longevity in alpha(1)-antitrypsin deficiency: the Copenhagen City Heart Study. 2003; Circulation. 107(5):747-52.
4. Perlmutter D H. The SEC receptor: a possible link between neonatal hepatitis in alpha 1-antitrypsin deficiency and Alzheimer's disease. Pediatric Research. 1994; 36(3):271-7.
5. Gollin, P A, Kalarai, R N, Eikelenboom, P. Rozemuller, A and Perry G, Alpha-1-antitrypsin and alpha-1-antichymotrypsin are in the lesions of Alzheimer's Disease. Neuroreport. 1992; 3(2):201-3.
6. Giometto, B, Argentiero, V, Sanson, F., Ongaro, G, and Tavolato, B., Acute-phase proteins in Alzheimer's Disease. European Neurology. 1988; 28(1):30-3.
7. Sun Y X. Minthon L. Wallmark A. Warkentin S. Blennow K. Janciauskiene S. Inflammatory markers in matched plasma and cerebrospinal fluid from patients with Alzheimer's disease. Dementia & Geriatric Cognitive Disorders. 2003; 16(3):136-44.
8. Schmechel, D. E., Burkhart, D. S., Ange, R. and Izard, M. K. Cholinergic axonal dystrophy and mitochondrial pathology in prosimian primates. Exp. Neurol. 1996; 142(1):11-127.
9. Corder, E. H., Saunders, A. M., Strittmatter, W. J., Schmechel, D., Gaskell, P., Small, G. W., et al. Gene dose of apolipoprotein E type 4 allele and the risk of Alzheimer disease in late onset families. Science 1993; 261(5123):921-923.
10. Pulliam J F. Jennings C D. Kryscio R J. Davis D G. Wilson D. Montine T J. Schmitt F A. Markesbery W R. Association of HFE mutations with neurodegeneration and oxidative stress in Alzheimer's disease and correlation with APOE. American Journal of Medical Genetics. Part B, Neuropsychiatric Genetics: the Official Publication of the International Society of Psychiatric Genetics. 119(1):48-53, 2003.
11. Schmechel, D. E., Tiller, M. O., Han, S.-H., Fail, P., Ange, R., Burkhart, D. S., Izard, M. K. Pattern of apolipoprotein-E immunoreactivity during brain aging. In: Apolipoprotein E and Alzheimer's Disease, A. D. Roses, K. Weisgraber, and Y. Christen, (eds) Y. Springer-Verlag, Berlin Heidelberg, 27-48, 1996.
12. Teckman J H. An J K. Blomenkamp K. Schmidt B. Perlmutter D. Mitochondrial autophagy and injury in the liver in alpha 1-antitrypsin deficiency. American Journal of Physiology—Gastrointestinal & Liver Physiology. 286(5):G851-62, 2004.
13. Talmud P J. Martin S. Steiner G. Flavell D M. Whitehouse D B. Nagl S. Jackson R. Taskinen M R. Frick M H. Nieminen M S. Kesaniemi Y A. Pasternack A. Humphries S E. Syvanne M. Diabetes Atherosclerosis Intervention Study Investigators. Progression of atherosclerosis is associated with variation in the alpha1-antitrypsin gene. Arteriosclerosis, Thrombosis & Vascular Biology. 23(4):644-9, 2003.
14. For example: Graziadei I. Weiss G. Egger C. Niederwieser D. Patsch J R. Vogel W. Modulation of iron metabolism in monocytic THP-1 cells and cultured human monocytes by the acute-phase protein alpha1-antitrypsin. Experimental Hematology. 1998; 26(11):1053-60.
15. Hayes V M. Genetic diversity of the alpha-1-antitrypsin gene in Africans identified using a novel genotyping assay. Human Mutation. 2003; 22(1):59-66.
16. Zandi, P P, Willour, V L, Huo, Y, Chellis, J, Potash, J B, MacKinnon D F, et al, Genome screen of a second wave of NIMH genetics initiative bipolar pedigrees: chromosomes 2, 11, 13, 14, and X. American Journal Human Genetics. 2003:119 B(1):69-76.
17. Corder, E. H., Saunders, A. M., Risch, N. J., Strittmatter, W. J., Schmechel, D. E., Gaskell, P. C., Jr., et al, Protective effect of apolipoprotein E type 2 allele for late onset Alzheimer disease. Nature Genetics 1994; 7:180-184.
18. Choi J. Malakowsky C A. Talent J M. Conrad C C. Gracy R W. Identification of oxidized plasma proteins in Alzheimer's disease. Biochemical & Biophysical Research Communications. 2002; 293(5):1566-70.
19. Moraga F. Janciauskiene S. Activation of primary human monocytes by the oxidized form of alpha1-antitrypsin. Journal of Biological Chemistry. 2000; 275(11):7693-700.
20. Mashiba S. Wada Y. Takeya M. Sugiyama A. Hamakubo T. Nakamura A. Noguchi N. Niki E. Izumi A. Kobayashi M. Uchida K. Kodama T. In vivo complex formation of oxidized alpha(1)-antitrypsin and LDL. Arteriosclerosis, Thrombosis & Vascular Biology. 2001; 21(11):1801-8.
21. Janciauskiene S. Moraga F. Lindgren S. C-terminal fragment of alpha1-antitrypsin activates human monocytes to a pro-inflammatory state through interactions with the CD36 scavenger receptor and LDL receptor. Atherosclerosis. 2001; 158(1):41-51.
22. Lomas D A. Lourbakos A. Cumming S A. Belorgey D. Hypersensitive mousetraps, alpha1-antitrypsin deficiency and dementia. Biochemical Society Transactions. 2002; 30(2):89-92.
23. Parfrey H. Mahadeva R. Lomas D A. Alpha(1)-antitrypsin deficiency, liver disease and emphysema. International Journal of Biochemistry & Cell Biology. 2003; 35(7):1009-14.
24. Liu Y. Choudhury P. Cabral C M. Sifers R N. Oligosaccharide modification in the early secretory pathway directs the selection of a misfolded glycoprotein for degradation by the proteasome. Journal of Biological Chemistry. 1999; 274(9):5861-7.
25. Janciauskiene S. Eriksson S. An interaction between Gemfibrozil and alpha 1-antitrypsin. Journal of Internal Medicine. 236(3):357-60, 1994.
26. Joslin G. Griffin G L. August A M. Adams S. Fallon R J. Senior R M. Perlmutter D H. The serpin-enzyme complex (SEC) receptor mediates the neutrophil chemotactic effect of alpha-1 antitrypsin-elastase complexes and amyloid-beta peptide. Journal of Clinical Investigation. 90(3):1150-4, 1992.
27. Huang H. Campbell S C. Nelius T. Bedford D F. Veliceasa D. Bouck N P. Volpert O V. Alpha1-antitrypsin inhibits angiogenesis and tumor growth. International Journal of Cancer. 112(6):1042-8, 2004.
28. Gil G. Hylemon P B. Suppression of cholesterol 7alpha-hydroxylase transcription and bile acid synthesis by an alpha1-antitrypsin peptide via interaction with alpha1-fetoprotein transcription factor. Journal of Biological Chemistry. 277(45):42973-80, 2002
29. Li W. Savinov A Y. Rozanov D V. Golubkov V S. Hedayat H. Postnova T I. Golubkova N V. Linli Y. Krajewski S. Strongin A Y. Matrix metalloproteinase-26 is associated with estrogen-dependent malignancies and targets alpha1-antitrypsin serpin. [Journal Article] Cancer Research. 64(23):8657-65, 2004.
30. Coraci I S. Husemann J. Berman J W. Hulette C. Dufour J H. Campanella G K. Luster A D. Silverstein S C. El-Khoury J B. CD36, a class B scavenger receptor, is expressed on microglia in Alzheimer's disease brains and can mediate production of reactive oxygen species in response to beta-amyloid fibrils. American Journal of Pathology. 160(1):101-12, 2002.
31. Novoradovsky A. Brantly M L. Waclawiw M A. Chaudhary P P. Ilara H. Qi L. Eissa N T. Barnes P M. Gabriele K M. Ehrnantraut M E. Rogliani P. Moss J. Endothelial nitric oxide synthase as a potential susceptibility gene in the pathogenesis of emphysema in alpha1-antitrypsin deficiency. American Journal of Respiratory Cell & Molecular Biology. 20(3):441-7, 1999.
32. Whiteman M. Szabo C. Halliwell B. Modulation of peroxynitrite- and hypochlorous acid-induced inactivation of alpha1-antiproteinase by mercaptoethylguanidine. British Journal of Pharmacology. 126(7): 1646-52, 1999.
33. Ikebe N. Akaike T. Miyamoto Y. Hayashida K. Yoshitake J. Ogawa M. Maeda H. Protective effect of S-nitrosylated alpha(1)-protease inhibitor on hepatic ischemia-reperfusion injury. Journal of Pharmacology & Experimental Therapeutics. 295(3):904-11, 2000.
34. Perlmutter D H. Liver injury in alpha1-antitrypsin deficiency: an aggregated protein induces mitochondrial injury. Journal of Clinical Investigation. 110(11):1579-83, 2002.
35. Mashiba S. Wada Y. Takeya M. Sugiyama A. Hamakubo T. Nakamura A. Noguchi N. Niki E. Izumi A. Kobayashi M. Uchida K. Kodama T. In vivo complex formation of oxidized alpha(1)-antitrypsin and LDL. Arteriosclerosis, Thrombosis & Vascular Biology. 21(11):1801-8, 2001.
36. Graziadei I. Weiss G. Bohm A. Werner-Felmayer G. Vogel W. Unidirectional upregulation of the synthesis of the major iron proteins, transferrin-receptor and ferritin, in HepG2 cells by the acute-phase protein alpha1-antitrypsin. Journal of Hepatology. 27(4):716-25, 1997
37. Weiss G. Graziadel I. Urbanek M. Grunewald K. Vogel W. Divergent effects of alpha 1-antitrypsin on the regulation of iron metabolism in human erythroleukaemic (K562) and myelomonocytic (THP-1) cells. Biochemical Journal. 319 (Pt 3):897-902, 1996
38. Graziadei I. Kahler C M. Wiedermann C J. Vogel W. The acute-phase protein alpha 1-antitrypsin inhibits transferrin-receptor binding and proliferation of human skin fibroblasts. Biochimica et Biophysica Acta. 1401(2):170-6, 1998.
39. Ikari Y. Mulvihill E. Schwartz S M. alpha 1-Proteinase inhibitor, alpha 1-antichymotrypsin, and alpha 2-macroglobulin are the antiapoptotic factors of vascular smooth muscle cells. Journal of Biological Chemistry. 276(15):11798-803, 2001.
40. Van Molle W. Libert C. Fiers W. Brouckaert P. Alpha 1-acid glycoprotein and alpha 1-antitrypsin inhibit TNF-induced but not anti-Fas-induced apoptosis of hepatocytes in mice. Journal of Immunology. 159(7):3555-64, 1997.
41. Kaup M. Dassler K. Reineke U. Weise C. Tauber R. Fuchs H. Processing of the human transferrin receptor at distinct positions within the stalk region by neutrophil elastase and cathepsin G. Biological Chemistry. 383(6):1011-20, 2002.
42. Perraud F. Besnard F. Labourdette G. Sensenbrenner M. Proliferation of rat astrocytes, but not of oligodendrocytes, is stimulated in vitro by protease inhibitors. International Journal of Developmental Neuroscience. 6(3):261-6, 1988.
43. Gollin P A. Kalaria R N. Eikelenboom P. Rozemuller A. Perry G. Alpha 1-antitrypsin and alpha 1-antichymotrypsin are in the lesions of Alzheimer's disease. Neuroreport. 3(2):201-3, 1992 Feb.
44. Carrell R. Corral J. What can Drosophila tell us about serpins, thrombosis and dementia? Bioessays. 26(1):1-5, 2004.
45. Green C. Brown G. Dafforn T R. Reichhart J M. Morley T. Lomas D A. Gubb D. Drosophila necrotic mutations mirror disease-associated variants of human serpins. Development. 130(7):1473-8, 2003.
46. Schmechel, D. E., Xu, P. T., Gilbert, J. R., Roses, A. D. Model of Genetic Susceptibility to Late-onset Alzheimer's Disease: Mice Transgenic for Human Apolipoprotein E Alleles. In: Mouse Models in the Study of Genetic Neurological Disorders, B. Popko (ed), Kluwer Academic/Plenum Press, New York, 1999.
47. Lee C. Maeng J S. Kocher J P. Lee B. Yu M H. Cavities of alpha(1)-antitrypsin that play structural and functional roles. Protein Science. 10(7):1446-53, 2001.
48. Elliott P R. Pei X Y. Dafforn T R. Lomas D A. Topography of a 2.0 A structure of alpha1-antitrypsin reveals targets for rational drug design to prevent conformational disease. Protein Science. 9(7):1274-81, 2000
49. Khatib H. Monoallelic expression of the protease inhibitor gene in humans, sheep, and cattle. Mammalian Genome. 16(1):50-8, 2005.
50. Hu C. Perlmutter D H. Regulation of alpha1-antitrypsin gene expression in human intestinal epithelial cell line caco-2 by HNF-1 alpha and HNF-4. American Journal of Physiology. 276(5 Pt 1):G1181-94, 1999.
51. Hu C. Perlmutter D H. Cell-specific involvement of HNF-1beta in alpha(1)-antitrypsin gene expression in human respiratory epithelial cells. American Journal of Physiology—Lung Cellular & Molecular Physiology. 282(4):L757-65, 2002.
52. El Khoury J B. Moore K J. Means T K. Leung J. Terada K. Toft M. Freeman M W. Luster A D. CD36 mediates the innate host response to beta-amyloid. Journal of Experimental Medicine. 197(12): 1657-66, 2003.
53. Mashiba S. Wada Y. Takeya M. Sugiyama A. Hamakubo T. Nakamura A. Noguchi N. Niki E. Izumi A. Kobayashi M. Uchida K. Kodama T. In vivo complex formation of oxidized alpha(1)-antitrypsin and LDL. Arteriosclerosis, Thrombosis & Vascular Biology. 21(11):1801-8, 2001.
54. Novoradovskaya N. Lee J. Yu Z X. Ferrans V J. Brantly M. Inhibition of intracellular degradation increases secretion of a mutant form of alpha1-antitrypsin associated with profound deficiency. Journal of Clinical Investigation. 101(12):2693-701, 1998.
55. Kotake Y, et al., Silvelstat, a neutrophil elastase inhibitor, attenuates neutrophil priming after hepatoenteric ischemia in rabbits, Shock, 23(2):156-160, 2005.
56. Fook J M et al, A serine protease inhibitor isolated from Tamarindus indica seeds and its effects on the release of human neutrophil elastase, Life Sciences. 76(25):2881-91, 2005.
57. Wieczorek M et al, Biochemical characterization of alpha-ketooxadiazole inhibitors of elastases, Arch Biochem Biophys 367(2):193-201, 1999.
58. Yoshimura Y et al., ONO-6818, a novel, potent neutrophil elastase inhibitor, reduces inflammatory mediators during simulated extracorporeal circulation. Ann Thoracic Surgery 76(4):1234-9, 2003.
59. Biduoard, J P, et al., SSR69071, an elastase inhibitor, reduces myocardial infarct size following ischemia-reperfusion injury, Eur J Pharmac, 461(1)49-52, 2003.
60. Wiedow O et al, Elafin: an elastase-specific inhibitor of human skin: purification, characterization, and complete amino acid sequence, J. Biol. Chem. 265:14791-14795, 1990.
61: Henriksen, P A et al, Gene delivery of the elastase inhibitor elafin protects macrophages from neutrophil elastase-mediated impairment of apoptotic cell recognition, FEBS Letters, 574(1-3):80-4, 2004.
62. Henriksen P A et al., Adenoviral gene delivery of elafin and secretoy leukocyte protease inhibitor attenuates the NF-kB-dependent inflammatory responses of human endothelial cells and macrophages to atherogenic stimuli, J. Immunol. 172:4535-4544, 2004.
63. Zeeuwen P L J M, Identification and sequence analysis of two new members of the SKALP/elafin and SPAI-2 gene family, J. Biol. Chem. 272:20471-20478, 1997.
64. Tremblay G M et al, Inhibition of human neutrophil elastase-induced acute lung injury in hamsters by recombinant human pre-elafin (trappin-2), Chest 121(2):582-588, 2002.
65. Van Wetering S. et al, Regulation of SLPI and elafin release from bronchial epithelial cells by neutrophil defensins, Am J. Physiol Lung Cell. Mole. Physiol. 278:L51-L58, 2000.
66. Petersen, R C et al., Practice parameter: early detection of mild dementia: mild cognitive impairment (an evidence-based review), Report of the Quality Standards Committee of the American Academy of Neurology, Neurology 2001; 56:1133-1142.
67. Knopman, D S et al., Practice parameter: diagnosis of dementia (an evidence-based review), Report of the Quality Standards Committee of the American Academy of Neurology, Neurology 2001; 56:1143-1153.
68. Zhang, M., et al., Differential expression of elafin in human normal mammary epithelial cells and carcinomas is regulated at the transcriptional level. Cancer Res. 55 (12), 2537-2541 (1995).
69. Thompson, R. C. and Ohlsson, K. Isolation, properties, and complete amino acid sequence of human secretory leukocyte protease inhibitor, a potent inhibitor of leukocyte elastase, Proc. Natl. Acad. Sci. U.S.A. 83 (18), 6692-6696 (1986).

We claim:

1. A method for predicting rate of progression of cognitive and/or behavioral decline in a subject with attention deficit disorder/attention deficit hyperactivity disorder, an affective disorder, mild cognitive impairment, dementia, or Alzheimer Disease, comprising:
determining types of alleles of alpha-1-antitrypsin (AAT) or AAT level in the subject with attention deficit disorder/attention deficit hyperactivity disorder, an affective disorder, mild cognitive impairment, dementia, or Alzheimer Disease;
using the determined types of alleles or AAT level as a factor to predict rate of progression of cognitive and/or behavioral decline in the subject.

2. A method for predicting vulnerability to or age of onset of attention deficit disorder/attention deficit hyperactivity disorder, an affective disorder, mild cognitive impairment, dementia, or Alzheimer Disease, comprising:
determining types of alleles of alpha-1-antitrypsin (AAT) or AAT level in a subject;
using the determined types of alleles and AAT level as a factor to predict vulnerability to or age of onset of Alzheimer Disease.

3. A method for predicting persons at risk of long-term nervous system injury, comprising:
determining types of alleles of alpha-1-antitrypsin (AAT) or AAT level in a subject who has been exposed to a neurotoxic or neuroinflammatory agent;
using the determined types of alleles or AAT level as a factor to predict persons at risk of long-term nervous system injury.

4. A method for predicting vulnerability of persons with pulmonary disease, liver disease, or coronary artery disease, to nervous system injury or cognitive or affective disorder, comprising:
determining types of alleles of alpha-1-antitrypsin (AAT) or AAT level in a subject with pulmonary disease, liver disease, or coronary artery disease;
using the determined types of alleles or AAT level as a factor to predict vulnerability of the subject to nervous system injury or cognitive or affective disorder.

5. The method of claim 1, 2, 3, or 4 wherein the types of alleles are determined by electrofocusing gels of AAT protein.

6. The method of claim 1, 2, 3, or 4 wherein the types of alleles are determined by polymerase chain reaction of AAT alleles.

7. The method of claim 1, 2, 3, or 4 wherein the type of at least one allele is determined by measuring anti-proteolytic activity or level of AAT, oxidized moieties of ATT, or fragments of AAT.

8. The method of claim 1 or 2 wherein the type of at least one allele is determined by ELISA methodology.

9. The method of claim 1 or 2 wherein the subject has a history of treated depression.

10. The method of claim 1 or 2 wherein the subject has depression.

11. The method of claim 1 or 2 wherein the subject has bipolar disease.

12. The method of claim 1 or 2 wherein the subject has generalized anxiety disorder.

13. The method of claim 1 or 2 wherein the subject has attention deficit disorder, attention deficit hyperactivity disorder, or both attention deficit and ADHD disorder, or dyslexia or developmental delay or school adjustment reaction.

14. The method of claim 1, 2, 3, or 4 wherein the subject has neuroimaging or clinical evidence for white matter disease.

15. The method of claim 1, 2, 3, or 4 wherein the subject has an S allele.

16. The method of claim 1, 2, 3, or 4 wherein the subject has a Z allele.

17. The method of claim 1, 2, 3, or 4 wherein the subject has an S and a Z allele or is homozygous for S or Z alleles.

18. The method of claim 1, 2, 3, or 4 wherein the subject has an I, P, F, V, G or null allele or other deficiency allele.

19. The method of claim 1 or 2 wherein the subject has cognitive dysfunction and is under 65 years of age.

20. The method of claim 19 wherein the subject has an S allele.

21. The method of claim 19 wherein the subject has a Z allele.

22. The method of claim 19 wherein the subject has an S and a Z allele or is homozygous for S or Z alleles

23. The method of claim 19 wherein the subject has an I, P, F, V, G or null allele or other deficiency allele.

24. The method of claim 1 wherein the subject presents with Alzheimer Disease, and wherein the subject is under 65 years of age.

25. The method of claim 24 wherein the subject has an S allele.

26. The method of claim 24 wherein the subject has a Z allele.

27. The method of claim 24 wherein the subject has an S and a Z allele or is homozygous for S or Z alleles

28. The method of claim 24 wherein the subject has an I, P, F, V, G or null allele or other deficiency allele.

29. The method of claim 1 wherein the subject presents with a diagnosis selected from the group consisting of non-amnesic mild cognitive impairment and frontotemporal dementia, and wherein the subject is under 65 years of age.

30. The method of claim 29 wherein the subject has an S allele.

31. The method of claim 29 wherein the subject has a Z allele.

32. The method of claim 29 wherein the subject has an S and a Z allele or is homozygous for S or Z alleles

33. The method of claim 29 wherein the subject has an I, P, F, V, G or null allele or other deficiency allele.

34. The method of claim 2 wherein a Z or null allele indicates an average age of onset of 55.

35. The method of claim 2 wherein an M or S allele indicates an average age of onset of 65.

36. The method of claim 1 wherein lower level of AAT is determined and indicates higher rates of progression of dementia.

37. The method of claim 2 further comprising determining the level of serum transferrin in the subject, wherein levels greater than ca. 280 mg/dl or third quartile and low AAT levels indicate an earlier age of onset of Alzheimer disease.

38. The method of claim 2 further comprising determining whether the subject carries an APOE2 allele or a C282Y allele of Hemochromatosis gene, wherein the APOE2 allele indicates an earlier age of onset of Alzheimer Disease and the C282Y allele indicates a later age of onset of Alzheimer Disease.

39. The method of claim 1 further comprising determining whether the subject carries a C282Y allele(s) of Hemochromatosis gene, wherein said allele indicates a slower rate of progression of Alzheimer Disease.

40. The method of claim 1 wherein the subject has an affective disorder which is bipolar disorder.

41. The method of claim 1 wherein the subject has an affective disorder which is anxiety disorder.

42. A method of delaying age of onset or progression rate of cognitive dysfunction in a subject at risk of developing cognitive dysfunction, comprising:
administering to the central nervous system of the subject AAT protein or a nucleic acid encoding AAT protein or C282Y hemochromatosis protein or a nucleic acid encoding C282Y hemochromatosis protein, whereby level or activity of AAT protein or hemochromatosis protein in the central nervous system of the subject is increased.

43. The method of claim 42 wherein the administering is intrathecal.

44. The method of claim 42 wherein a protein is administered.

45. The method of claim 42 wherein a nucleic acid is administered.

46. The method of claim 42 wherein the administering is directly into the brain of the subject.

47. The method of claim 42 wherein the administering is intracerebroventricular.

48. A method of delaying age of onset or progression rate of cognitive dysfunction in a subject comprising:
administering to the blood stream or liver of a subject at risk of developing Alzheimer Disease AAT protein or a nucleic acid encoding AAT protein or C282Y hemochromatosis protein or a nucleic acid encoding C282Y hemochromatosis protein, whereby level or activity of AAT protein or C282Y protein in the blood stream or liver of the subject is increased.

49. The method of claim 48 wherein AAT protein is administered.

50. The method of claim 48 wherein a nucleic acid is administered.

51. The method of claim 48 wherein the administering is to the blood stream.

52. The method of claim 48 wherein the administering is to the liver.

53. A method of delaying age of onset or diminishing progression rate of cognitive dysfunction in a subject who has an AAT deficiency phenotype, comprising:
administering to the subject thiamine supplements or a low carbohydrate diet, whereby age of onset of cognitive dysfunction is delayed or progression rate of cognitive dysfunction is diminished.

54. A method of diminishing progression rate of cognitive dysfunction in a subject who has attention deficit disorder, an affective disorder, or Alzheimer Disease, comprising:
administering to the subject thiamine supplements or a low carbohydrate diet, whereby age of onset of cognitive dysfunction is delayed or progression rate of cognitive dysfunction is diminished.

55. A method of screening for candidate drugs for treatment of cognitive dysfunction, comprising:
contacting a test substance with an AAT protein encoded by an S, Z, I, P, F, V, G, or null allele;
determining activity of the AAT protein;
identifying the test substance as a candidate drug for treatment of cognitive dysfunction if it increases activity of the AAT protein.

56. A method of screening for candidate drugs for treatment of cognitive dysfunction, comprising:
contacting a test substance with a cell which has an AAT-deficient phenotype;
determining activity of AAT protein in the cell;
identifying the test substance as a candidate drug for treatment of cognitive dysfunction if it increases total amount of activity of AAT in the cell.

57. A method of screening for candidate drugs for treatment of cognitive dysfunction, comprising:
contacting a test substance with a cell which encodes an AAT protein;
determining amount of AAT protein expressed in the cell;
identifying the test substance as a candidate drug for treatment of cognitive dysfunction if it increases expression or activity of AAT in the cell.

58. A method of screening for candidate drugs for treatment of cognitive dysfunction, comprising:
contacting a test substance with a first AAT protein encoded by an S, Z, I, P, F, V, G, or a null allele;
contacting the AAT with cells selected from the group consisting of monocytes, macrophages, astroglia, microglia, oligodendroglia, choroid plexus cells, cerebral endothelial cells, and progenitors thereof;
determining in said cells a parameter selected from the group consisting of ferritin concentration, transferrin (Tf) receptor concentration, endocytosis of Tf receptor and ligand, free iron concentration, transferrin receptor release or shedding, IRE/IRP activity, and IRE/IRP modulated targets;
identifying the test substance as a candidate drug for treatment of cognitive dysfunction if it modulates the effect of the first AAT protein on the parameter so that it is more similar to the effect of an AAT protein encoded by an M allele than the effect of the first protein on the parameter in the absence of the test substance.

59. A method of screening for candidate drugs for treatment of cognitive dysfunction, comprising:
contacting a test substance with a first cell which has an AAT-deficient phenotype;
contacting the AAT from the first cell with cells selected from the group consisting of monocytes, macrophages, astroglia, microglia, oligodendroglia, choroid plexus cells, cerebral endothelial cells, and progenitors thereof;
determining in said cells a parameter selected from the group consisting of ferritin concentration, transferrin (Tf) receptor concentration, endocytosis of Tf receptor and ligand, free iron concentration, transferrin receptor release or shedding, IRE/IRP activity, and IRE/IRP modulated targets;
identifying the test substance as a candidate drug for treatment of cognitive dysfunction if it modulates effect of the AAT protein from the first cell on the parameter so that it is more similar to the effect of an AAT protein from a cell without a deficiency allele in the absence of the test substance.

60. A method of screening for candidate drugs for treatment of cognitive dysfunction, comprising:
contacting a test substance with a cell which encodes an AAT protein;
contacting the AAT from the cell with a cell selected from the group consisting of monocytes, macrophages, astroglia, microglia, oligodendroglia, choroid plexus cells, cerebral endothelial cells, and progenitors thereof;
determining in said cells a parameter selected from the group consisting of ferritin concentration, transferrin (Tf) receptor concentration, endocytosis of Tf receptor and ligand, free iron concentration, transferrin receptor release or shedding, IRE/IRP activity, and IRE/IRP modulated targets;
identifying the test substance as a candidate drug for treatment of cognitive dysfunction if it enhances the effect of the AAT protein on the parameter.

61. A method of screening for candidate drugs for treatment of cognitive dysfunction, comprising:
contacting a test substance with a first AAT protein encoded by an S, Z, or other deficiency allele or a null allele;
determining carbonyl or oxidized or nitrosylated groups on the first AAT protein or activity of the first AAT protein;
identifying the test substance as a candidate drug for treatment of cognitive dysfunction if it modulates amount of carbonyl or oxidized or nitrosylated groups on the AAT protein and/or activity of AAT protein.

62. A method for screening for candidate drugs for treatment of cognitive dysfunction, comprising:
contacting a test substance with a cell which has an AAT-deficient phenotype;
determining amount of carbonyl or oxidized or nitrosylated groups on the AAT protein in the cell or the activity of the AAT protein;
identifying the test substance as a candidate drug for treatment of cognitive dysfunction if it modulates the amount of carbonyl or oxidized or nitrosylated groups on the AAT protein and/or activity of AAT protein.

63. A method for screening for candidate drugs for treatment of cognitive dysfunction, comprising:
contacting a test substance with a cell which encodes an AAT protein;
determining amount of carbonyl or oxidized or nitrosylated groups on the AAT protein expressed in the cell or activity of the AAT protein;
identifying the test substance as a candidate drug for treatment of cognitive dysfunction if it modulates amount of carbonyl or oxidized or nitrosylated groups of AAT and/or activity of AAT.

64. A method of screening for candidate drugs for treatment of cognitive dysfunction, comprising:
contacting a test substance with a first AAT protein encoded by an S, Z, or other deficiency allele or a null allele;
determining monocyte and/or macrophage activating activity of the AAT protein;
identifying the test substance as a candidate drug for treatment of cognitive dysfunction if it modulates the effect of the first AAT protein on the monocyte and/or macrophage activating activity of the AAT protein if it modulates the effect of the first AAT protein on the parameter so that it is more similar to the effect of an AAT protein encoded by an M allele than the effect of the first protein on the parameter in the absence of the test substance.

65. A method of screening for candidate drugs for treatment of cognitive dysfunction, comprising:
contacting a test substance with a cell which has an AAT-deficient phenotype;
determining monocyte and/or macrophage activating activity of the AAT protein;
identifying the test substance as a candidate drug for treatment of cognitive dysfunction if it increases monocyte and/or macrophage activating activity of the AAT protein.

66. A method for screening for candidate drugs for treatment of cognitive dysfunction, comprising:
contacting a test substance with a cell which encodes an AAT protein;
determining monocyte and/or macrophage activating activity of the AAT protein;
identifying the test substance as a candidate drug for treatment of cognitive dysfunction if it increases monocyte and/or macrophage activating activity of the AAT protein.

67. A method of screening for candidate drugs for treatment of cognitive dysfunction, comprising:
contacting a test substance with an AAT protein encoded by an S, Z, or other rare deficiency allele or a null allele;
determining amount of carboxyl terminal fragment of AAT (C-36);
identifying the test substance as a candidate drug for treatment of cognitive dysfunction if it modulates amount of the C-36.

68. A method of screening for candidate drugs for treatment of cognitive dysfunction;
contacting a test substance with a cell which has an AAT-deficient phenotype;
determining amount of carboxyl terminal fragment of AAT (C-36);
identifying the test substance as a candidate drug for treatment of cognitive dysfunction if it modulates amount of the C-36.

69. A method for screening for candidate drugs for treatment of cognitive dysfunction;
contacting a test substance with a cell which encodes an AAT protein;
determining amount of carboxyl terminal fragment of AAT (C-36);
identifying the test substance as a candidate drug for treatment of cognitive dysfunction if it modulates amount of the C-36.

70. A method of delaying age of onset or progression rate of cognitive dysfunction in a subject having or at risk of developing attention deficit disorder/attention deficit hyperactivity disorder, an affective disorder, mild cognitive impairment, dementia, or Alzheimer Disease, comprising:
administering to the central nervous system of the subject an agent for inhibiting neutrophil elastase activity or expression selected from the group consisting of: an antibody which specifically binds to neutrophil elastase, an antisense molecule comprising at least 18 contiguous nucleotides which are complementary to mRNA encoding neutrophil elastase, FR901277, SC-37698, SC-39026, SKALP/elafin, SLPI, sivelestat (ONO-5046; Elaspol; C₂₀H₂₁N₂O₇S.4H2O.Na.), ONO-6818 (C₂₃H₂₈N₆O₄, molecular weight: 452.51)), FR901277 (C₄₇H₆₃N₉O₁₃, molecular weight: 961), SC-37698, SC-39026, and SSR69071 (2-(9-(2-Piperidinoethoxy)-4-oxo-4H-pyrido[1,2-a]pyrimidin-2-yloxy-methyl)-4-(1-methylethyl)-6-methoxy-1,2-benzisothiazol-3(2H)-one-1,1-dioxide), whereby age of onset or progression rate of cognitive dysfunction in the subject is delayed or diminished.

71. The method of claim 69 wherein the administering is intrathecal.

72. The method of claim 69 wherein the antibody is administered.

73. The method of claim 69 wherein the antisense molecule is administered.

74. The method of claim 69 wherein the administering is directly into the brain of the subject.

75. The method of claim 69 wherein the administering is intracerebroventricular.

76. The method of claim 69 wherein an agent selected from the group consisting of FR901277, SC-37698, SC-39026, SKALP/elafin, pre-elafin, SLPI, sivelestat (ONO-5046; Elaspol; C₂₀H₂₁N₂O₇S.4H2O.Na.), ONO-6818 (C₂₃H₂₈N₆O₄, molecular weight: 452.51)), FR901277 (C₄₇H₆₃N₉O₁₃, molecular weight: 961), SC-37698, SC-39026, and SSR69071 (2-(9-(2-Piperidinoethoxy)-4-oxo-4H-pyrido[1,2-a]pyrimidin-2-yloxy-methyl)-4-(1-methylethyl)-6-methoxy-1,2-benzisothiazol-3(2H)-one-1,1-dioxide) is administered.

77. A method of screening for candidate drugs for treatment of cognitive dysfunction, comprising:
contacting a test substance with a neutrophil elastase protein;
determining activity of the neutrophil elastase protein;
identifying the test substance as a candidate drug for treatment of cognitive dysfunction if it decreases activity of the neutrophil elastase protein.

78. A method of screening for candidate drugs for treatment of cognitive dysfunction, comprising:
contacting a test substance with a cell which expresses a human neutrophil elastase;
determining activity of human neutrophil elastase protein in the cell;
identifying the test substance as a candidate drug for treatment of cognitive dysfunction if it decreases total amount of activity of the neutrophil elastase in the cell.

79. A method of delaying age of onset or diminishing progression rate of cognitive dysfunction in a subject who has an AAT deficiency phenotype, comprising:
administering lithium to the subject, whereby age of onset of cognitive dysfunction is delayed or progression rate of cognitive dysfunction is diminished.

80. A method of diminishing progression rate of cognitive dysfunction in a subject who has or who is at risk of developing attention deficit disorder/attention deficit hyperactivity disorder, an affective disorder, or Alzheimer Disease, comprising:
administering lithium to the subject, whereby progression rate of cognitive dysfunction is diminished.

81. The method of claim 44 wherein the protein is ATT.

82. The method of claim 44 wherein the protein is C282Y hemochromatosis protein

83. The method of claim 45 wherein the nucleic acid encodes ATT.

84. The method of claim 45 wherein the nucleic acid encodes C282Y hemochromatosis protein.

85. The method of claim 49 wherein the protein is ATT.

86. The method of claim 49 wherein the protein is C282Y hemochromatosis protein

87. The method of claim 50 wherein the nucleic acid encodes ATT.

88. The method of claim 50 wherein the nucleic acid encodes C282Y hemochromatosis protein.
